(19) United States
(12) Patent Application Publication
(10) Pub. No.: US 2025/0171499 A1
(43) Pub. Date: May 29, 2025

(54) PHARMACEUTICAL FORMULATIONS AND METHODS FOR THE TREATMENT OF METABOLIC AND LIVER DISORDERS

(71) Applicant: Viking Therapeutics, Inc., San Diego, CA (US)

(72) Inventors: Brian Lian, Rancho Santa Fe, CA (US); Geoffrey E. Barker, Carlsbad, CA (US); Maureen Barnes, San Diego, CA (US); Kader Yagiz, San Diego, CA (US)

(21) Appl. No.: 18/729,459

(22) PCT Filed: Jan. 11, 2023
(86) PCT No.: PCT/US2023/010565

§ 371 Date: Jul. 16, 2024

Related U.S. Application Data
(60) Provisional application No. 63/300,538, filed on Jan. 18, 2022.

(51) Int. Cl. C07K 14/00; A61K 38/00; A61K 47/10; A61K 47/14; A61K 47/22

(52) U.S. Cl. CPC C07K 14/001; A61K 47/10; A61K 47/14; A61K 47/22; A61K 38/00

(57) ABSTRACT

Disclosed herein are formulations of small molecule GIP/GLP-1 dual receptor agonists and uses thereof.

### BACKGROUND

### Field

The present disclosure relates generally to the field of treatments for metabolic disorders and fatty liver diseases. More specifically, the present disclosure relates to the field of formulations of small molecule drugs for the treatment of diseases including non-alcoholic steatohepatitis (NASH), non-alcoholic fatty liver disease (NAFLD).

### Description of the Related Art

Incretin peptides glucose-dependent insulinotropic polypeptide (GIP) and glucagon-like peptide-1 (GLP-1) are metabolic hormones. GIP and GLP-1 are both secreted within minutes of nutrient ingestion and facilitate the rapid disposal of ingested nutrients. Both peptides share common actions on islet β-cells acting through structurally distinct yet related receptors. Incretin-receptor activation leads to glucose-dependent insulin secretion, induction of β-cell proliferation, and enhanced resistance to apoptosis. GIP also promotes energy storage via direct actions on adipose tissue. In contrast, GLP-1 exerts glucoregulatory actions via slowing of gastric emptying and glucose-dependent inhibition of glucagon secretion. GLP-1 also promotes satiety and sustained GLP-1-receptor activation is associated with weight loss in both preclinical and clinical studies.

Non-alcoholic fatty liver disease (NAFLD) is the hepatic manifestation of metabolic syndrome and is the most common cause of chronic liver disease. NAFLD may progress to liver inflammation, fibrosis, cirrhosis and even hepatocellular carcinoma. GIP/GLP-1 dual receptor agonists have been developed for treating NAFLD, non-alcoholic steatohepatitis (NASH), diabetes, obesity, and other diseases. However, the use of GIP/GLP-1 dual receptor agonists is associated with nausea, vomiting, and/or diarrhea. For example, clinical trials of a GIP/GLP1 dual receptor agonist compound found that tolerability at high doses was limited by gastrointestinal adverse events. The dose limitation associated with gastrointestinal adverse events may prevent dosing to the desired effective dose, may compromise patient compliance with treatment, and may limit the effectiveness of the treatment regimen. Therefore, a need exists for formulations of novel GIP/GLP1 dual agonist compounds that can be used to treat fatty liver diseases and other diseases and disorders.

### SUMMARY

Some embodiments disclosed herein include a pharmaceutical formulation for administration to a subject in need thereof, wherein the pharmaceutical formulation comprises: a pharmaceutically acceptable carrier, a pharmaceutically acceptable diluent, and any combination of the foregoing; and a therapeutically effective dosage of a compound having the structure of formula I:

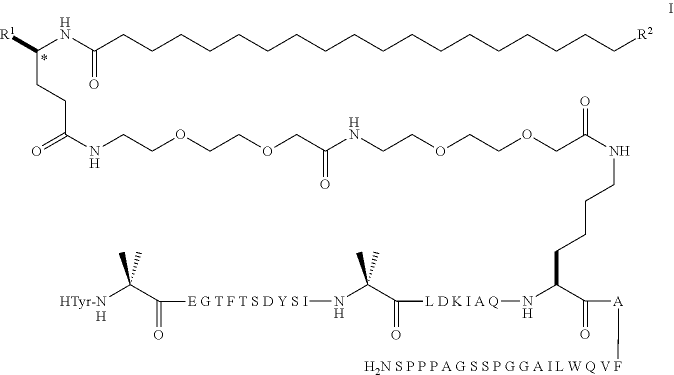

or a pharmaceutically acceptable salt thereof, wherein:
R¹ is selected from the group consisting of —C(═O)(OZ¹), —P(═O)(X)(Y) and a 5-10 membered heteroaryl containing 1-2 heteroatoms selected from N, O and S optionally substituted with 1-2 R⁷ independently selected from halogen, C₁₋₆ alkyl, haloC₁₋₆ alkyl, haloC₁₋₆ alkoxy, —OR⁵, C₃₋₁₀ cycloalkyl, C₆₋₁₀ aryl, 5-10 membered heteroaryl and 5-10 membered heterocyclyl;
R² is selected from the group consisting of —C(═O)(OZ²), —P(═O)(X)(Y) and a 5-10 membered heteroaryl containing 1-2 heteroatoms selected from N, O and S optionally substituted with 1-2 R⁷ independently selected from halogen, C₁₋₆ alkyl, haloC₁₋₆ alkyl, haloC₁₋₆ alkoxy, —OR⁵, C₃₋₁₀ cycloalkyl, C₆₋₁₀ aryl, 5-10 membered heteroaryl and 5-10 membered heterocyclyl;
each R⁷ may be independently selected from the group consisting of halogen, C₁₋₆ alkyl, haloC₁₋₆ alkyl, haloC₁₋₆ alkoxy, C₁₋₆ alkoxy, C₃₋₁₀ cycloalkyl, C₆₋₁₀ aryl, 5-10 membered heteroaryl and 5-10 membered heterocyclyl;
X and Y may each be independently selected from the group consisting of —OR⁴, NR⁵R⁶, C₁₋₆ alkyl and haloC₁₋₆ alkyl;
each R⁴ may be independently selected from the group consisting of hydrogen, C₁₋₆ alkyl, haloC₁₋₆ alkyl, C₆₋₁₀ aryloxy and C₆₋₁₀ aryl alkoxy;
each R⁵ may be independently hydrogen or C₁₋₆ alkyl;
each R⁶ may be independently hydrogen or C₁₋₆ alkyl; and
Z¹ and Z² may each be independently selected from the group consisting of hydrogen, C₁₋₆ alkyl, haloC₁₋₆ alkyl, haloC₁₋₆ alkoxy, C₁₋₆ alkoxy, C₃₋₁₀ cycloalkyl and C₆₋₁₀ aryl,
with the proviso that at least one of Z¹ and Z² is not hydrogen.

Other embodiments disclosed herein include the pharmaceutically acceptable carrier that comprises at least 10% by weight of propylene glycol.

Other embodiments disclosed herein include the pharmaceutically acceptable diluent that is a pH buffer. In some embodiments, the pharmaceutically acceptable diluent is present in the formulation at a weight percentage of equal to or more than about 20%.

Other embodiments disclosed herein include a method of preventing, treating, or ameliorating one or more fatty liver diseases in a subject, by administering the pharmaceutical formulations disclosed herein to a subject in need thereof. The fatty liver diseases include but are not limited to steatosis, non-alcoholic steatohepatitis (NASH), non-alcoholic fatty liver disease (NAFLD).

Other embodiments disclosed herein include a method of preventing, treating, or ameliorating one or disease or disorders in a subject, by administering the pharmaceutical formulations disclosed herein to a subject in need thereof. In some embodiments, said disease or disorder is liver fibrosis, renal fibrosis, biliary fibrosis, pancreatic fibrosis, nonalcoholic steatohepatitis, non-alcoholic fatty liver disease, chronic kidney disease, diabetic kidney disease, primary sclerosing cholangitis, primary biliary cirrhosis, or idiopathic fibrosis. In some embodiments, said disease or disorder is a metabolic disorder or a metabolic syndrome. In some embodiments, said disease or disorder is atherosclerosis, diabetes, hyperglycemic diabetes, type 2 diabetes mellitus, dyslipidemia, hypercholesterolemia, hyperlipidemia, hypertension, hypoglycemia, obesity, or prader-willi syndrome.

### DETAILED DESCRIPTION

In some embodiments, pharmaceutical formulations are provided for administration to a subject in need thereof. Various embodiments of these pharmaceutical formulations include a pharmaceutically acceptable carrier, a pharmaceutically acceptable excipient, a pharmaceutically acceptable diluent, and any combination of the foregoing. Some embodiments of the pharmaceutical formulations include a therapeutically effective dosage of a compound, or a pharmaceutically acceptable salt thereof, as described elsewhere herein. Some embodiments of the pharmaceutical formulations are administered for the prevention, treatment, or amelioration of one or more fatty liver diseases in the subject. The fatty liver diseases include but are not limited to steatosis, non-alcoholic steatohepatitis (NASH), non-alcoholic fatty liver disease (NAFLD).

In some embodiments, the pharmaceutical formulations include compounds that are non-macrocyclic functionalized peptides that act as GIP/GLP-1 dual receptor agonists. Various embodiments of these compounds include compounds having the structure of formula I as described above or pharmaceutically acceptable salts thereof. The structure of formula I encompasses all stereoisomers and racemic mixtures, including the following structure and mixtures thereof:

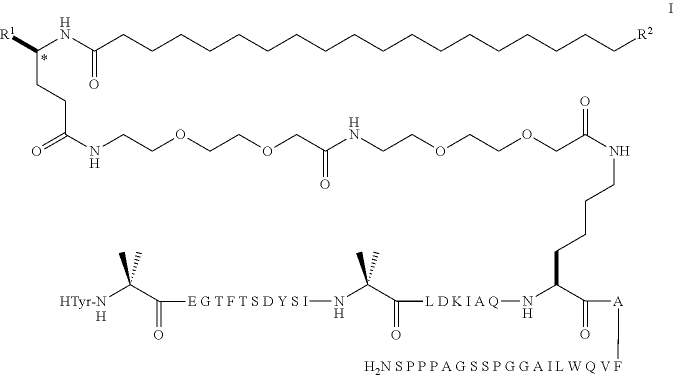

In some embodiments of compounds of formula I:
R¹ is selected from the group consisting of —C(═O)(OZ¹), —P(═O)(X)(Y) and a 5-10 membered heteroaryl containing 1-2 heteroatoms selected from N, O and S optionally substituted with 1-2 R⁷ independently selected from halogen, C₁₋₆ alkyl, haloC₁₋₆ alkyl, haloC₁₋₆ alkoxy, —OR⁵, C₃₋₁₀ cycloalkyl, C₆₋₁₀ aryl, 5-10 membered heteroaryl and 5-10 membered heterocyclyl;
R² is selected from the group consisting of —C(═O)(OZ²), —P(═O)(X)(Y) and a 5-10 membered heteroaryl containing 1-2 heteroatoms selected from N, O and S optionally substituted with 1-2 R⁷ independently selected from halogen, C₁₋₆ alkyl, haloC₁₋₆ alkyl, haloC₁₋₆ alkoxy, —OR⁵, C₃₋₁₀ cycloalkyl, C₆₋₁₀ aryl, 5-10 membered heteroaryl and 5-10 membered heterocyclyl;
each R⁷ may be independently selected from the group consisting of halogen, C₁₋₆ alkyl, haloC₁₋₄ alkyl, haloC₁₋₆ alkoxy, C₁₋₆ alkoxy, C₃₋₁₀ cycloalkyl, C₆₋₁₀ aryl, 5-10 membered heteroaryl and 5-10 membered heterocyclyl;
X and Y may each be independently selected from the group consisting of —OR⁴, NR⁵R⁶, C₁₋₆ alkyl and haloC₁₋₆ alkyl;
each R⁴ may be independently selected from the group consisting of hydrogen, C₁₋₆ alkyl, haloC₁₋₆ alkyl, C₆₋₁₀ aryloxy and C₆₋₁₀ aryl alkoxy;
each R⁵ may be independently hydrogen or C₁₋₆ alkyl;
each R⁶ may be independently hydrogen or C₁₋₆ alkyl; and
Z¹ and Z² may each be independently selected from the group consisting of hydrogen, C₁₋₆ alkyl, haloC₁₋₆ alkyl, haloC₁₋₆ alkoxy, C₁₋₆ alkoxy, C₃₋₁₀ cycloalkyl and C₆-10 aryl, with the proviso that at least one of Z¹ and Z² is not hydrogen.

Some embodiments of compounds of formula I include compounds having the structure of formula I-a:

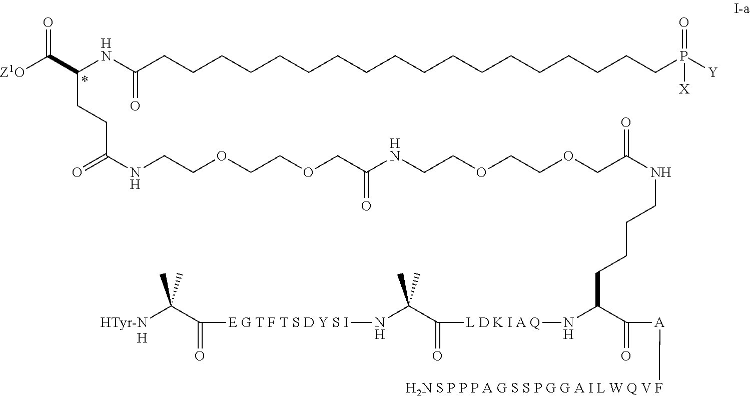

or pharmaceutically acceptable salts thereof.

In some embodiments of compounds of formula I-a or their pharmaceutically acceptable salts; Z¹ is selected from hydrogen, C₁₋₆ alkyl, haloC₁₋₆ alkyl, haloC₁₋₆ alkoxy, C₁₋₆ alkoxy, C₃₋₁₀ cycloalkyl and C₆₋₁₀ aryl; and X and Y each are —OR⁴.

In some embodiments of compounds of formula I-a or their pharmaceutically acceptable salts; Z¹ is selected from hydrogen, haloC₁₋₆ alkoxy and C₁₋₆ alkoxy; and each R may be independently selected from hydrogen, C₆₋₁₀ aryloxy and C₆₋₁₀ aryl alkoxy.

In some embodiments of compounds of formula I-a or their pharmaceutically acceptable salts; Z¹ is hydrogen and each R may be independently hydrogen or C₆₋₁₀ aryl alkoxy.

In some embodiments of compounds of formula I-a or their pharmaceutically acceptable salts; each R⁴ is hydrogen.

In some embodiments of compounds of formula I-a or their pharmaceutically acceptable salts; Z¹ is hydrogen and each R⁴ is hydrogen.

Some embodiments of compounds of formula I include compounds having the structure of formula I-b:

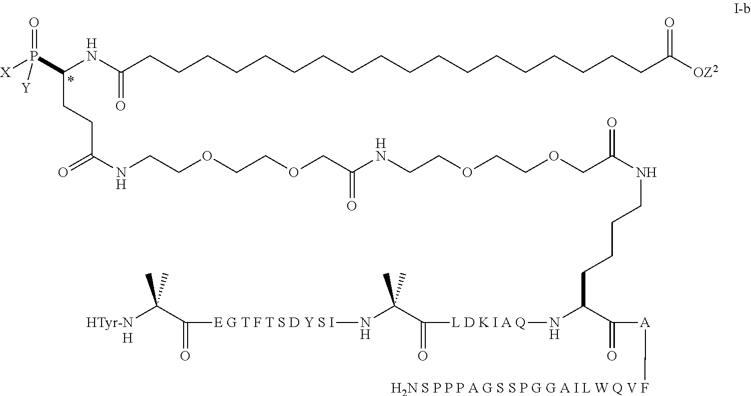

or pharmaceutically acceptable salts thereof.

In some embodiments of compounds of formula I-b or their pharmaceutically acceptable salts; Z² is selected from hydrogen, C₁₋₆ alkyl, haloC₁₋₆ alkyl, haloC₁₋₆ alkoxy, C₁₋₆ alkoxy, C₃₋₁₀ cycloalkyl and C₆₋₁₀ aryl; and X and Y each are —OR⁴.

In some embodiments of compounds of formula I-b or their pharmaceutically acceptable salts; Z² is selected from hydrogen, haloC₁₋₆ alkoxy and C₁₋₆ alkoxy; and each R⁴ may be independently selected from hydrogen, C₆₋₁₀ aryloxy and C₆₋₁₀ aryl alkoxy.

In some embodiments of compounds of formula I-b or their pharmaceutically acceptable salts; Z² is hydrogen and each R⁴ may be independently hydrogen or C₆-10 aryl alkoxy.

In some embodiments of compounds of formula I-b or their pharmaceutically acceptable salts; each R⁴ is hydrogen.

In some embodiments of compounds of formula I-b or their pharmaceutically acceptable salts; Z² is hydrogen and each R⁴ is hydrogen.

Some embodiments of compounds of formula I include compounds having the structure of formula I-c:

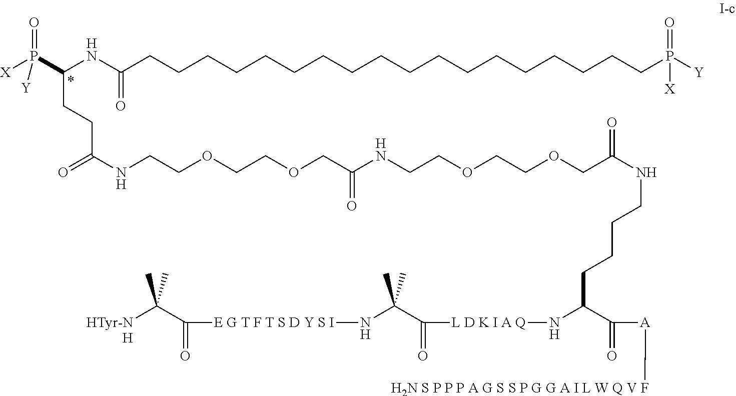

or pharmaceutically acceptable salts thereof.

In some embodiments of compounds of formula I-c or their pharmaceutically acceptable salts; X and Y each are —OR⁴.

In some embodiments of compounds of formula I-c or their pharmaceutically acceptable salts; each R⁴ may be independently selected from hydrogen, C₆₋₁₀ aryloxy and C₆₋₁₀ aryl alkoxy.

In some embodiments of compounds of formula I-c or their pharmaceutically acceptable salts; each R⁴ is hydrogen.

Some embodiments include a compound having the structure selected from the group consisting of:

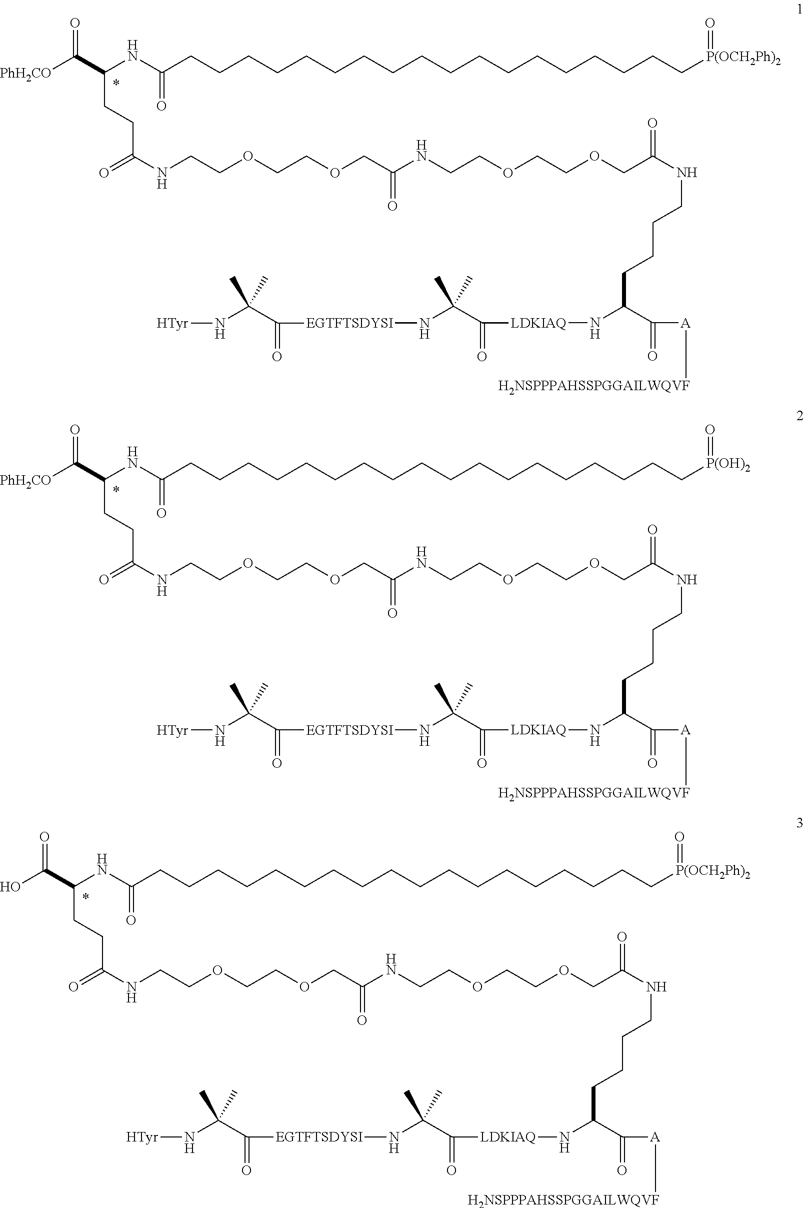

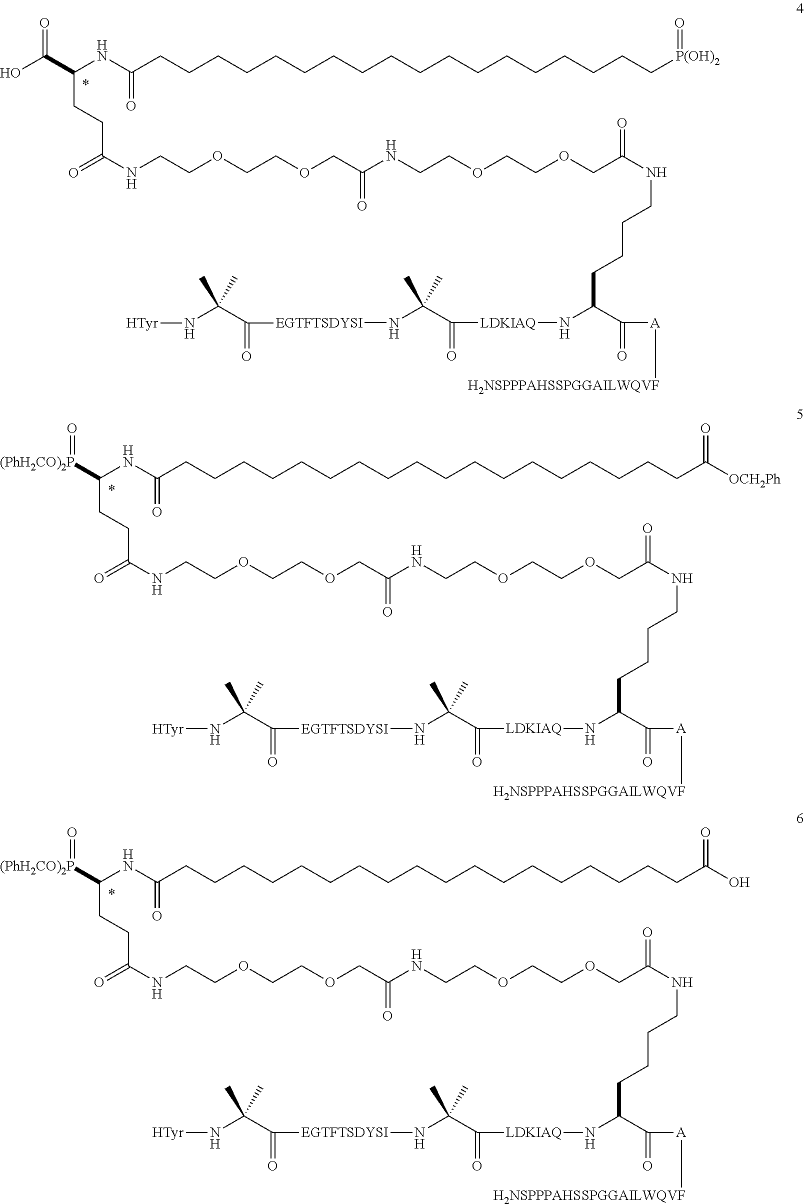

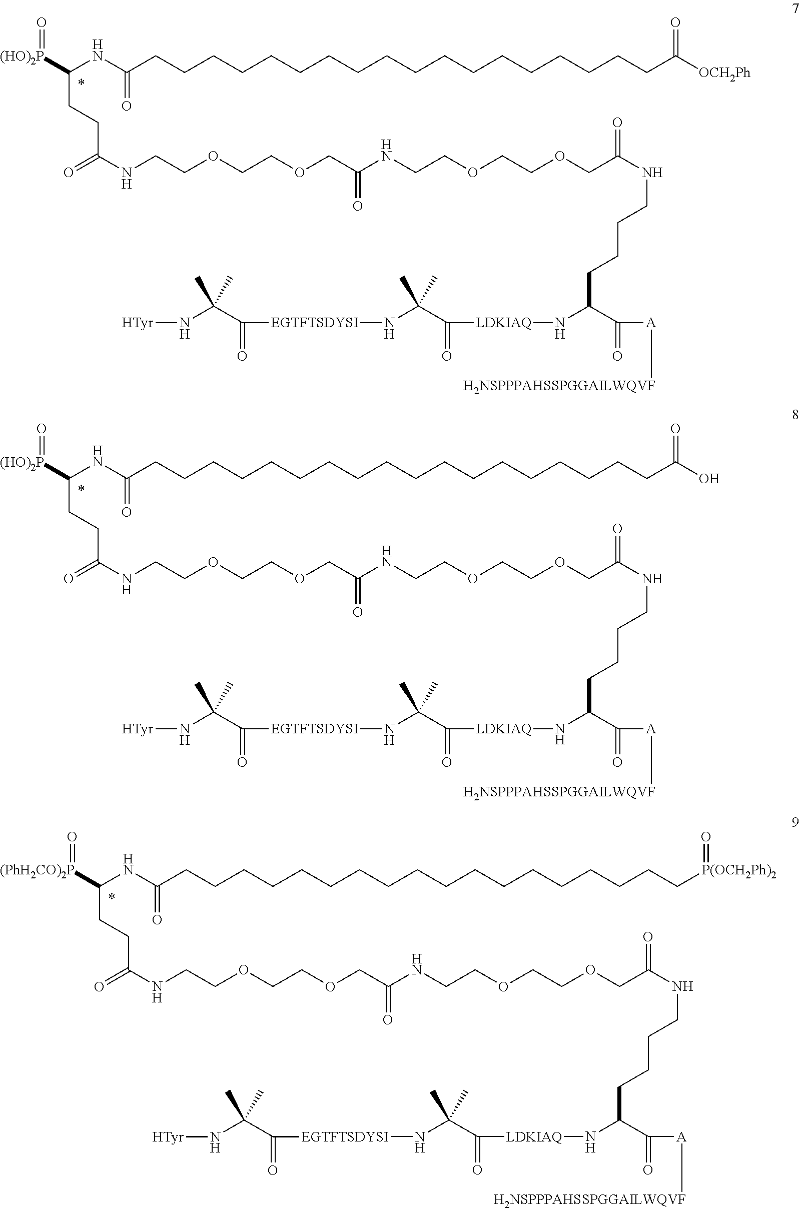

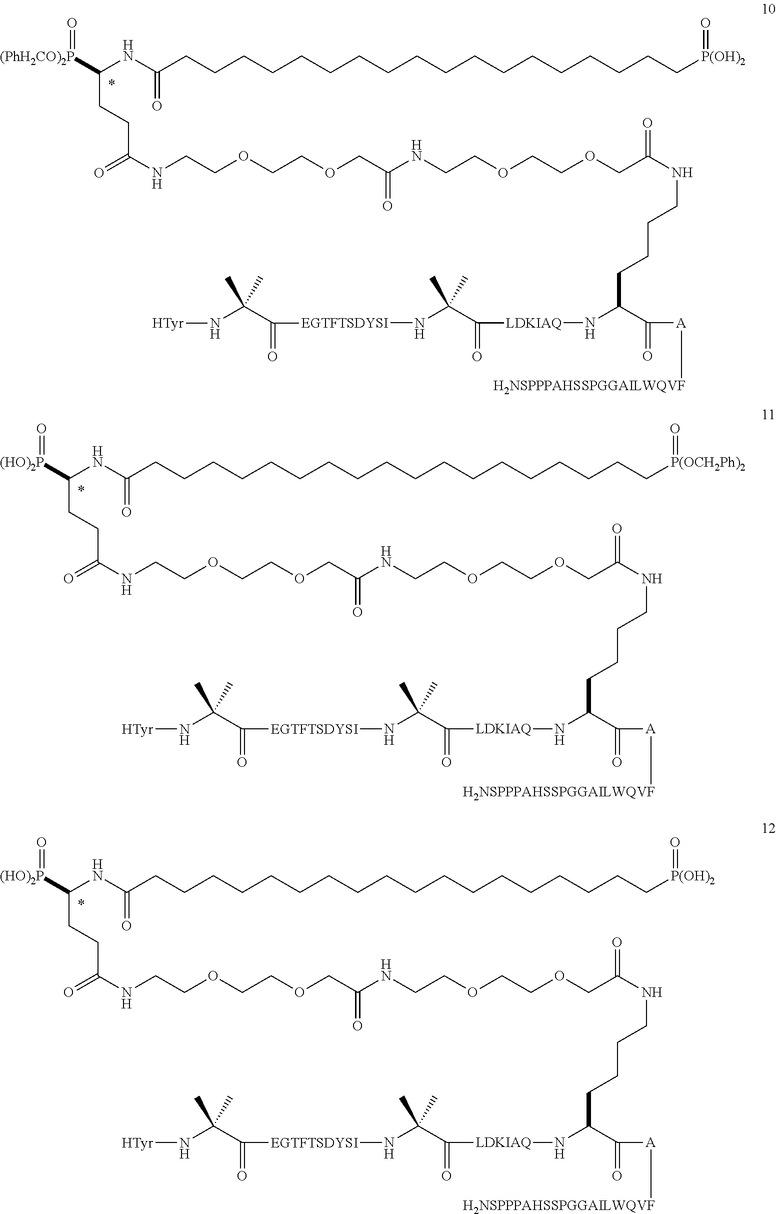

and pharmaceutically acceptable salts thereof.

Some embodiments include a compound wherein “*” indicates a chiral carbon with “S” configuration.

Some embodiments include a compound wherein “*” indicates a chiral carbon with “R” configuration.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

The pharmaceutical formulations include at least one pharmaceutically acceptable carrier. The term “pharmaceutically acceptable carrier,” as used herein, is given its ordinary meaning to those skilled in the art. In some embodiments, the pharmaceutically acceptable carrier comprises propylene glycol. In some embodiments, the pharmaceutically acceptable carrier is present in the formulation at a weight percentage of equal to or more than about: 1%, 5%, 10%, 15%, 20%, 25%, 30%, 31%, 32%, 33%, 34%, 35%, 36%, 37%, 38% 39%, 40%, 41%, 42%, 43%, 44%, 45%, 46%, 47%, 48%, 49%, 50%, 60%, 70%, 80%, 90%, 95%, or ranges including and/or spanning the aforementioned values. In some embodiments, the pharmaceutically acceptable carrier is present in the formulation at a weight percentage from about 10% to 90% by weight. In some embodiments, the pharmaceutically acceptable carrier is present in the formulation at a weight percentage from about 30% to 50%.

The pharmaceutical formulations may include at least one pharmaceutically acceptable diluent. The term “pharmaceutically acceptable diluent,” as used herein, is given its ordinary meaning to those skilled in the art. In some embodiments, the pharmaceutically acceptable diluent comprises saline or sterilized water. In some embodiments, the pharmaceutically acceptable diluent comprises a pH buffer. In some embodiments, the pharmaceutically acceptable diluent is a pH buffer selected from tartrate, citrate, acetate, 2-(N-morpholino)ethanesulfonic acid (MES), piperazine-N,N′-bis(2-ethanesulfonic acid (PIPES), 3-(N-morpholino)propanesulfonic acid (MOPS), 2-[[1,3-dihydroxy-2-(hydroxymethyl)propan-2-yl]amino]ethanesulfonic acid (TES), 4-(2-hydroxyethyl)-1-piperazineethanesulfonic acid (HEPES), 3-[N-tris(hydroxymethyl) methylamino]-2-hydroxypropanesulfonic acid (TAPSO), N-[tris(hydroxymethyl)methyl]glycine (Tricine), tris(hydroxymethyl)aminomethane (Tris), 2-(bis(2-hydroxyethyl)amino) acetic acid (Bicine), tris(hydroxymethyl) methylamino]propanesulfonic acid (TAPS), N-cyclohexyl-2-aminoethanesulfonic acid (CHES), phosphate, borate, and any combination of the foregoing in a suitable solvent (e.g., water). In some embodiments, the pharmaceutically acceptable diluent has a pH of about: 1, 2, 3, 4, 5, 6, 7, 8, 9, 10, 11, 12, 13, 14, or ranges including and/or spanning the aforementioned values. In some embodiments, the pharmaceutically acceptable diluent has a pH from about 3 to 7. In some embodiments, the pharmaceutically acceptable diluent has a pH from about 4 to 6.8. In some embodiments, the concentration of the pharmaceutically acceptable diluent is about: 0.01 mM, 0.05 mM, 0.1 mM, 0.5 mM, 1 mM, 5 mM, 6 mM, 7 mM, 8 mM, 9 mM, 10 mM, 11 mM, 12 mM, 13 mM, 14 mM, 15 mM, 20 mM, 25 mM, 30 mM, 35 mM, 40 mM, 45 mM, 50 mM, 70 mM, 100 mM, 200 mM, 500 mM, 1000 mM, or ranges including and/or spanning the aforementioned values. In some embodiments, the concentration of the pharmaceutically acceptable diluent is from about 1 to 50 mM. In some embodiments, the concentration of the pharmaceutically acceptable diluent is from about 8 to 12 mM. In some embodiments, the pharmaceutically acceptable diluent is present in the formulation at a weight percentage of equal to or more than about: 1%, 5%, 10%, 20%, 30%, 40%, 50%, 53%, 55%, 57%, 59%, 60%, 61%, 63%, 65%, 67%, 70%, 75%, 80%, 90%, 95%, or ranges including and/or spanning the aforementioned values. In some embodiments, the pharmaceutically acceptable diluent is present in the formulation at a weight percentage from about 20% to 95% by weight. In some embodiments, the pharmaceutically acceptable diluent is present in the formulation at a weight percentage from about 50% to 70%.

In some embodiments, the pharmaceutical formulations comprise propylene glycol and a pH buffer. In some embodiments, the pH buffer comprises tartrate in a suitable solvent (e.g., water). In some embodiments, the pH buffer comprises citrate in a suitable solvent (e.g., water). In some embodiments, the pH buffer comprises acetate in a suitable solvent (e.g., water). In some embodiments, the pH buffer comprises 2-(N-morpholino)ethanesulfonic acid (MES) in a suitable solvent (e.g., water). In some embodiments, the pH buffer comprises piperazine-N,N′-bis(2-ethanesulfonic acid (PIPES) in a suitable solvent (e.g., water). In some embodiments, the pH buffer has a pH of about: 1, 2, 3, 4, 5, 6, 7, 8, 9, 10, 11, 12, 13, 14, or ranges including and/or spanning the aforementioned values. In some embodiments, the pH buffer has a pH from about 3 to 7. In some embodiments, the pH buffer has a pH from about 4 to 6.8. In some embodiments, the pH buffer has a pH from about 5 to 6.8. In some embodiments, the concentration of the pH buffer is about: 0.01 mM, 0.05 mM, 0.1 mM, 0.5 mM, 1 mM, 5 mM, 6 mM, 7 mM, 8 mM, 9 mM, 10 mM, 11 mM, 12 mM, 13 mM, 14 mM, 15 mM, 20 mM, 25 mM, 30 mM, 40 mM, 50 mM, 70 mM, 100 mM, 200 mM, 500 mM, 1000 mM, or ranges including and/or spanning the aforementioned values. In some embodiments, the concentration of the pH buffer is from about 1 to 50 mM. In some embodiments, the concentration of the pH buffer is from about 8 to 12 mM. In some embodiments, propylene glycol is present in the formulation at a weight percentage of equal to or more than about 10% and the pH buffer is present in the formulation at a weight percentage of equal to or more than about 20%. In some embodiments, the propylene glycol is present in the formulation at a weight percentage from about 10% to 90%. In some embodiments, the propylene glycol is present in the formulation at a weight percentage from about 20% to 70%. In some embodiments, the propylene glycol is present in the formulation at a weight percentage from about 30% to 50%. In some embodiments, the pH buffer is present in the formulation at a weight percentage from about 20% to 95%. In some embodiments, the pH buffer is present in the formulation at a weight percentage from about 30% to 80%. In some embodiments, pH buffer is present in the formulation at a weight percentage from about 50% to 70%. In some embodiments, the pH of the formulation is about 1, 2, 3, 4, 5, 6, 7, 8, 9, 10, 11, 12, 13, 14, or ranges including and/or spanning the aforementioned values. In some embodiments, the formulation has a pH from about 3 to 7. In some embodiments, the formulation has a pH from about 4 to 6.8. In some embodiments, the formulation has a pH from about 5 to 6.8.

The pharmaceutical formulations include a therapeutically effective dosage. The term “therapeutically effective dosage,” as used herein, is dependent on the subject and disease state being treated, the severity of the affliction, the manner and schedule of administration and the judgment of the prescribing physician. In some embodiments, the therapeutically effective dosage may be a daily dosage from about 0.0125 mg/kg to about 120 mg/kg or more of body weight, from about 0.025 mg/kg or less to about 70 mg/kg, from about 0.05 mg/kg to about 50 mg/kg of body weight, or from about 0.075 mg/kg to about 10 mg/kg of body weight. Thus, for administration to a 70 kg person, the dosage range would be from about 0.88 mg per day to about 8000 mg per day, from about 1.8 mg per day or less to about 7000 mg per day or more, from about 3.6 mg per day to about 6000 mg per day, from about 5.3 mg per day to about 5000 mg per day, or from about 11 mg to about 3000 mg per day. In some embodiments, the therapeutically effective dosage is from about 0.001 mg/kg, 0.005 mg/kg, 0.01 mg/kg, 0.05 mg/kg, 0.1 mg/kg, 0.12 mg/kg, 0.14 mg/kg, 0.15 mg/kg, 0.16 mg/kg, 0.18 mg/kg, 0.19 mg/kg, 0.2 mg/kg, 0.21 mg/kg, 0.22 mg/kg, 0.24 mg/kg, 0.25 mg/kg, 0.26 mg/kg, 0.28 mg/kg, 0.3 mg/kg, 0.5 mg/kg, 1 mg/kg, 2 mg/kg, 3 mg/kg, 5 mg/kg, 10 mg/kg, 25 mg/kg, 50 mg/kg, 100 v, 200 mg/kg, 500 mg/kg, or ranges including and/or spanning the aforementioned values. In some embodiments, the therapeutically effective dosage is from about 0.01 mg/kg to about 5 mg/kg. In some embodiments, the therapeutically effective dosage is from about 0.05 mg/kg to about 1 mg/kg. In some embodiments, the therapeutically effective dosage is from about 0.15 mg/kg to about 0.25 mg/kg.

The pharmaceutical formulations have a half life in the blood of the subject when they are administered. In some embodiments, the half life is equal to or greater than about: 5 h, 10 h, 20 h, 40 h, 60 h, 80 h, 90 h, 100 h, 110 h, 115 h, 118 h, 120 h, 122 h, 125 h, 128 h, 130 h, 150 h, 180 h, 200 h, 250 h, 300 h, 500 h, or ranges including and/or spanning the aforementioned values. In some embodiments, the half life is from about 40 h to 300 h. In some embodiments, the half life is from about 60 h to 150 h.

The pharmaceutical formulations can be administered with a route of administration including, but not limited to, enteral, intravenous, oral, intraarticular, intramuscular, subcutaneous, intraperitoneal, epidural, intranasal, topical, intrapulmonary, vaginal, rectal, transdermal, and transmucosal. In some embodiments, the route of administration selected from the group consisting of enteral, intravenous, oral, intraarticular, intramuscular, subcutaneous, intraperitoneal, epidural, transdermal, and transmucosal. In some embodiments, the pharmaceutical formulations are administered subcutaneously. In some embodiments, the pharmaceutical formulations are administered intravenously. In some embodiments, the pharmaceutical formulations are administered orally.

The pharmaceutical formulations can be provided in a dosage form. In some embodiments, the dosage form is selected from a solid form and a liquid form. The solid dosage forms include tablets, capsules, granules and bulk powders. The liquid dosage forms include solutions, emulsions, and suspensions. In some embodiments, the dosage form is a solid form. In some embodiments, the dosage form is a liquid form.

In some embodiments, the pharmaceutical formulations include at least one pharmaceutically-acceptable excipient(s). The term “pharmaceutically acceptable excipient,” as used herein, includes but is not limited to solvents, dispersants, coatings, antimicrobial bacterial agents, adjuvants, isotonic and absorption delaying agents and the like. In some embodiments, the pharmaceutically-acceptable excipients include sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers and surfactants, such as the TWEENS; wetting agents, such sodium lauryl sulfate; coloring agents; flavoring agents; tableting agents, stabilizers; antioxidants; preservatives such as benzalkonium chloride, PHMB, chlorobutanol, thimerosal, phenylmercuric, and phenylmercuric nitrate; tonicity adjustors such as sodium chloride, potassium chloride, mannitol and glycerin; vehicles such as polyvinyl alcohol, povidone, hydroxypropyl methyl cellulose, poloxamers, carboxymethyl cellulose, and hydroxyethyl cellulose; and pyrogen-free water. In some embodiments, the pharmaceutically-acceptable excipient(s) are selected based on the route of administration and can include solid or liquid fillers, diluents, hydrotropies, surface-active agents, and encapsulating substances. For example, in the case of intravenous administration, excipients may include gelatin; carbohydrates such as dextrose, mannitol, and dextran; and antioxidants such as sodium bisulfite, acetone sodium bisulfite, sodium formaldehyde, sulfoxylate, thiourea, and EDTA. In some embodiments, the pharmaceutically-acceptable excipient(s) include antimicrobial agents such as phenylmercuric nitrate, thimerosal, benzethonium chloride, benzalkonium chloride, phenol, cresol, and chlorobutanol. Additional examples of suitable pharmaceutically-acceptable excipient(s) are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, each of which are incorporated herein by reference in their entirety.

In some embodiments, the pharmaceutical formulations are administered to a subject that is a mammal.

In some embodiments, the pharmaceutical formulations are administered to a subject that is a human.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “polyethylene glycol” refers to the formula

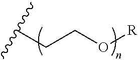

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “(C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A})SO₂R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆-10 aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆-10 aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a “natural amino acid side chain” refers to the side-chain substituent of a naturally occurring amino acid. Naturally occurring amino acids have a substituent attached to the α-carbon. Naturally occurring amino acids include Arginine, Lysine, Aspartic acid, Glutamic acid, Glutamine, Asparagine, Histidine, Serine, Threonine, Tyrosine, Cysteine, Methionine, Tryptophan, Alanine, Isoleucine, Leucine, Phenylalanine, Valine, Proline, and Glycine.

As used herein, a “non-natural amino acid side chain” refers to the side-chain substituent of a non-naturally occurring amino acid. Non-natural amino acids include β-amino acids (β³ and β²), Homo-amino acids, Proline and Pyruvic acid derivatives, 3-substituted Alanine derivatives, Glycine derivatives, Ring-substituted Phenylalanine and Tyrosine Derivatives, Linear core amino acids and N-methyl amino acids. Exemplary non-natural amino acids are available from Sigma-Aldridge, listed under “unnatural amino acids & derivatives.” See also, Travis S. Young and Peter G. Schultz, “Beyond the Canonical 20 Amino Acids: Expanding the Genetic Lexicon,” J. Biol. Chem. 2010 285: 11039-11044, which is incorporated by reference in its entirety.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

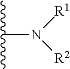

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

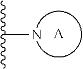

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

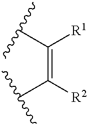

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

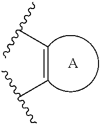

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

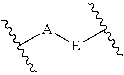

includes the substituent being oriented such that the A is attached at the leftmost attachment point of the molecule as well as the case in which A is attached at the rightmost attachment point of the molecule.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

In one embodiment, the methods disclosed herein may include constructing a 39-amino acid peptide backbone using solid-phase peptide synthesis techniques to provide intermediate (II). The peptide backbone includes two PEG₂ amide linkers. The method includes an amide coupling reaction between the amine of the terminal PEG₂ amide of intermediate (II) and an appropriately substituted carboxylic acid (III) to provide the resin-bound intermediate (IV). In one embodiment, the method involves subjecting intermediate (IV) to hydrolysis under acidic conditions followed by purification to yield the final product (I). (Scheme 1).

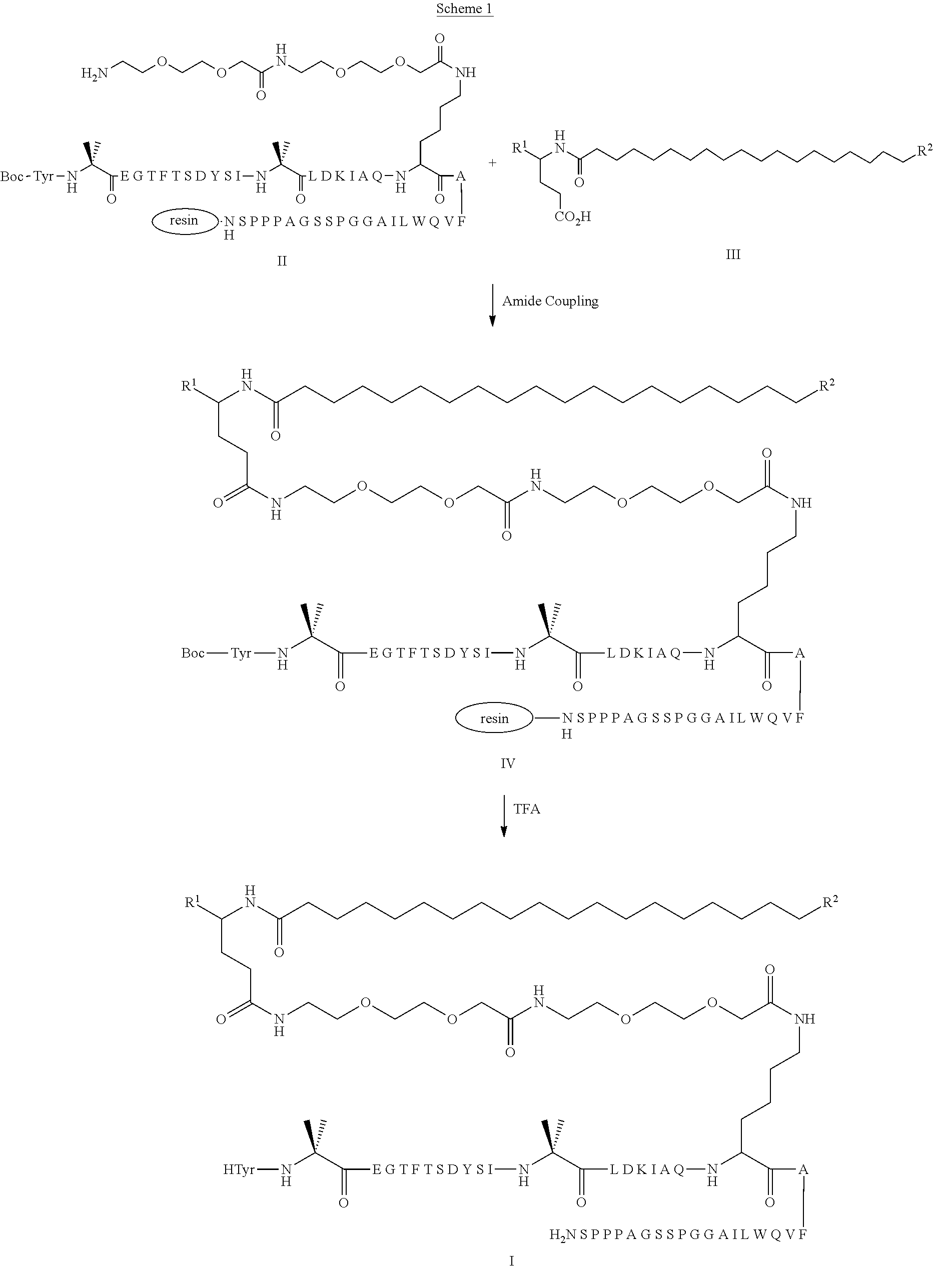

The above example schemes are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

The pharmaceutical formulations disclosed herein can be prepared using standard pharmaceutical formulation techniques, such as those disclosed in: Remington's The Science and Practice of Pharmacy, 21st Ed., Lippincott Williams & Wilkins (2005), Gilman et al. (Eds.) (1990); Goodman and Gilman's: The Pharmacological Basis of Therapeutics, 8th Ed., Pergamon Press, each of which is incorporated herein by reference in its entirety. In some embodiments, the pharmaceutical formulations can be prepared as unit dosage forms. Techniques and compositions for preparing unit dosage forms are described in: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004), each of which is incorporated herein by reference in its entirety.

### Methods of Treatment

The pharmaceutical formulations disclosed herein include compounds or their tautomers and/or pharmaceutically acceptable salts thereof that can effectively act as GIP/GLP1 dual receptor agonists. The pharmaceutical formulations further comprise one or more pharmaceutically acceptable carriers and one or more pharmaceutically acceptable diluents.

Some embodiments provide a method of preventing, treating, or ameliorating one or more fatty liver diseases in a subject. In some embodiments, the method includes administering one or more of the pharmaceutical formulations disclosed herein to a subject in need thereof.

Some embodiments provide a method preventing, treating, or ameliorating steatosis, non-alcoholic steatohepatitis and non-alcoholic fatty liver disease. In some embodiments, the method includes administering one or more of the pharmaceutical formulations disclosed herein to a subject in need thereof.

In some embodiments, the method of administering one or more of the pharmaceutical formulations disclosed herein results in the prevention, treatment, or amelioration, of a fibrosis, fibrotic condition, or fibrotic symptoms.

In some embodiments, the pharmaceutical formulations described herein can be used to treat a host of conditions arising from fibrosis or inflammation, and specifically including those associated with myofibroblast differentiation. Exemplary conditions include but are not limited to progressive liver fibrosis (alcoholic, viral, autoimmune, metabolic and hereditary chronic disease), renal fibrosis (e.g., resulting from chronic inflammation, infections or type II diabetes), lung fibrosis (idiopathic or resulting from environmental insults including toxic particles, sarcoidosis, asbestosis, hypersensitivity pneumonitis, bacterial infections including tuberculosis, medicines, etc.), interstitial fibrosis, systemic scleroderma (autoimmune disease in which many organs become fibrotic), macular degeneration (fibrotic disease of the eye), pancreatic fibrosis (resulting from, for example, alcohol abuse and chronic inflammatory disease of the pancreas), fibrosis of the spleen (from sickle cell anemia, other blood disorders), cardiac fibrosis (resulting from infection, inflammation and hypertrophy), mediastinal fibrosis, myelofibrosis, endomyocardial fibrosis, retroperitoneal fibrosis, progressive massive fibrosis, nephrogenic systemic fibrosis, diabetic nephropathy, non-alcoholic steatohepatitis, primary sclerosing cholangitis, corneal fibrosis, liver cirrhosis, fibrotic complications of surgery, chronic allograft vasculopathy and/or chronic rejection in transplanted organs, ischemic reperfusion injury associated fibrosis, injection fibrosis, cirrhosis, diffuse parenchymal lung disease, post-vasectomy pain syndrome, and rheumatoid arthritis diseases or disorders.

In some embodiments, the method of administering one or more of the pharmaceutical formulations disclosed herein results in the reduction in the amount of extracellular matrix proteins present in one or more tissues of said subject.

In some embodiments, the method of administering one or more of the pharmaceutical formulations disclosed herein results in the reduction in the amount of collagen present in one or more tissues of said subject.

In some embodiments, the method of administering one or more of the pharmaceutical formulations disclosed herein results in the reduction in the amount of Type I, Type Ia, or Type III collagen present in one or more tissues of said subject.

Some embodiments provide a method of preventing, treating, or ameliorating one or more of liver fibrosis, renal fibrosis, biliary fibrosis, pancreatic fibrosis, nonalcoholic steatohepatitis, non-alcoholic fatty liver disease, chronic kidney disease, diabetic kidney disease, primary sclerosing cholangitis, primary biliary cirrhosis, or idiopathic fibrosis in a subject. In some embodiments, the method includes administering one or more of the pharmaceutical formulations disclosed herein to a subject in need thereof.

Some embodiments provide a method of preventing, treating, or ameliorating one or more of nonalcoholic steatohepatitis, non-alcoholic fatty liver disease, chronic kidney disease, diabetic kidney disease, primary sclerosing cholangitis, or primary biliary cirrhosis in a subject. In some embodiments, the method includes administering one or more of the pharmaceutical formulations disclosed herein to a subject in need thereof.

Some embodiments provide a method of preventing, treating, or ameliorating one or more metabolic disorders or metabolic syndromes. In some embodiments, said disease or disorder is atherosclerosis, diabetes, hyperglycemic diabetes, type 2 diabetes mellitus, dyslipidemia, hypercholesterolemia, hyperlipidemia, hypertension, hypoglycemia, obesity, or prader-willi syndrome. In some embodiments, the method includes administering one or more of the pharmaceutical formulations disclosed herein to a subject in need thereof.

In some embodiments, the method of administering one or more of the pharmaceutical formulations disclosed herein results in the activation of a glucose-dependent insulinotropic polypeptide (GIP) receptor. In some embodiments, the method of administering one or more of the pharmaceutical formulations disclosed herein results in the activation of a glucagon-like peptide-1 (GLP-1) receptor. In some embodiments, the method of administering one or more of the pharmaceutical formulations disclosed herein results in the activation of the GIP receptor and the GLP-1 receptor.

Some embodiments include co-administering a pharmaceutical formulation and/or a compound, or pharmaceutically acceptable salt thereof, described herein, with an additional medicament. By “co-administration,” it is meant that the two or more agents may be found in the patient's bloodstream at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered subcutaneously, another being administered orally and another being administered i.v.

To further illustrate this disclosure, the following examples are included. The examples should not, of course, be construed as specifically limiting the disclosure. Variations of these examples within the scope of the claims are within the purview of one skilled in the art and are considered to fall within the scope of the disclosure as described, and claimed herein. The reader will recognize that the skilled artisan, armed with the present disclosure, and skill in the art is able to prepare and use the disclosure without exhaustive examples. The following examples will further describe the present disclosure, and are used for the purposes of illustration only, and should not be considered as limiting.

### EXAMPLES

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present disclosure were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the disclosure. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the disclosure.

The following abbreviations have the indicated meanings:
Aib=aminoisobutyric acid
Bn=benzyl
Boc=tert-butoxycarbonyl
Bu=butyl
DMF=dimethylformamide
EDC=1-ethyl-3-(3-dimethylaminopropyl)carbodiimide
Et=ethyl
HATU=hexafluorophosphate azabenzotriazole tetramethyl uranium
HBTU=hexafluorophosphate benzotriazole tetramethyl uranium
HMDS=hexamethyldisilazane
HPLC=high-performance liquid chromatography
Me=methyl
NaHMDS=sodium hexamethyldisilazide
NMR=nuclear magnetic resonance
PCC=pyridinium chlorochromate
PEG=polyethylene glycol
Ph=phenyl
tBu=tert-butyl
TFA=trifluoroacetic acid
THF=tetrahydrofuran
TMS=trimethylsilyl

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### Synthesis of Intermediate 1 (INT 1)

Methyl 7-bromoheptanoate is treated with triphenylphosphine to form the corresponding phosphonium salt. The salt is treated with one equivalent of NaHMDS to make an ylide, which is reacted immediately in a Wittig reaction with the aldehyde from PCC oxidation of 12-bromo-1-dodecanol. The resulting bromo alkene is hydrogenated, and treated with dibenzyl phosphite in weak base to form a phosphonate ester. Hydrolysis of the methyl carboxylate provides desired INT 1 having a terminal carboxylic acid and a dibenzyl phosphonate.

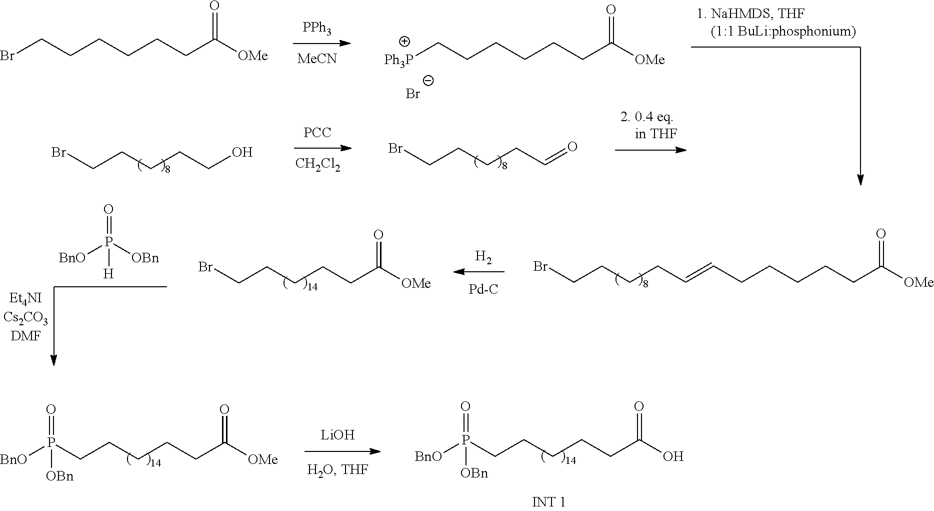

### Synthesis of Intermediate 2 (INT 2)

Docosanedioic acid is coupled to benzyl alcohol with EDC-HCl and DMAP in THF to give INT 2 as the monobenzyl ester.

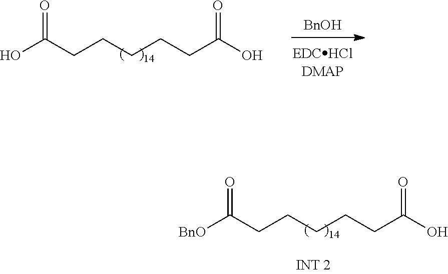

### Synthesis of Intermediate 3 (INT 3)

t-Butyl 4-hydroxybutanoate undergoes a Swern oxidation to give an aldehyde. The aldehyde is condensed with (R)-1-amino-2-methoxy-1-phenylethane to form an imine. Addition of the lithium salt of diethyl phosphite in THF generates an α-aminophosphonate, which undergoes hydrogenolysis to cleave the N-alkyl group and provide INT 3 with a free primary amine, a t-butyl ester, and a diethyl phosphonate ester. The optical purity of INT 3 was confirmed to be at least 96% by 1H NMR through Mosher's amide analysis.

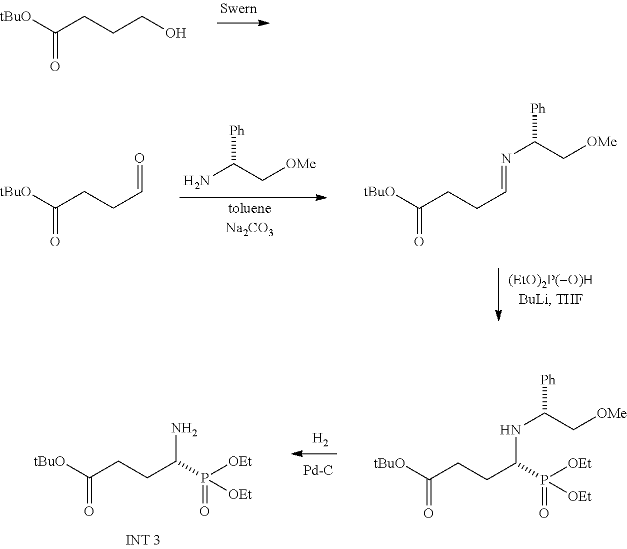

### Synthesis of Intermediate 4 (INT 4)

INT 1 is coupled with the 1-t-butyl ester of D-glutamic acid in the presence of HATU and triethylamine in DMF to provide INT 4.

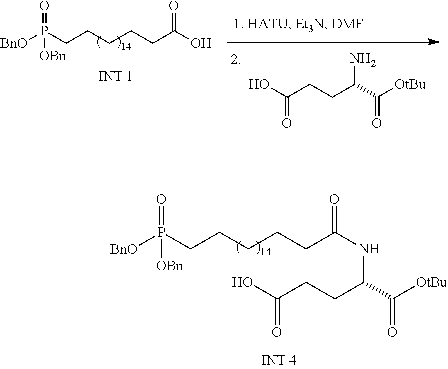

### Synthesis of Intermediate 5 (INT 5)

INT 2 is coupled with INT 3 in the presence of HATU and triethylamine in DMF to prepare a new amide linkage. Cleavage of the ethyl phosphonate esters with TMS-Br gives the free phosphonic acid. Re-esterification with a large excess of the benzyl ester of N,N′-diisopropylcarbamimidic acid provides the corresponding dibenzyl phosphonate. The t-butyl ester is cleaved with TFA to provide INT 5.

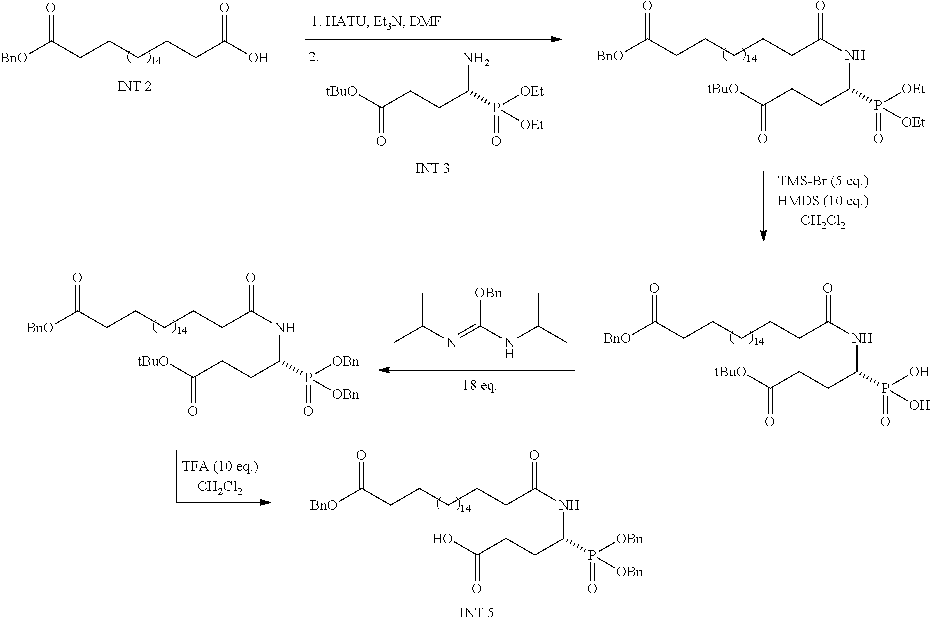

### Synthesis of Intermediate 6 (INT 6)

INT 1 is coupled with INT 3 in the presence of HATU and triethylamine in DMF to provide a new amide linkage. Cleavage of the benzyl and ethyl phosphonate esters with TMS-Br gives both free phosphonic acids. Re-esterification with a large excess of the benzyl ester of N,N′-diisopropylcarbamimidic acid provides the corresponding tetrabenzyl diphosphonate ester. The t-butyl ester is cleaved with TFA to give INT 6.

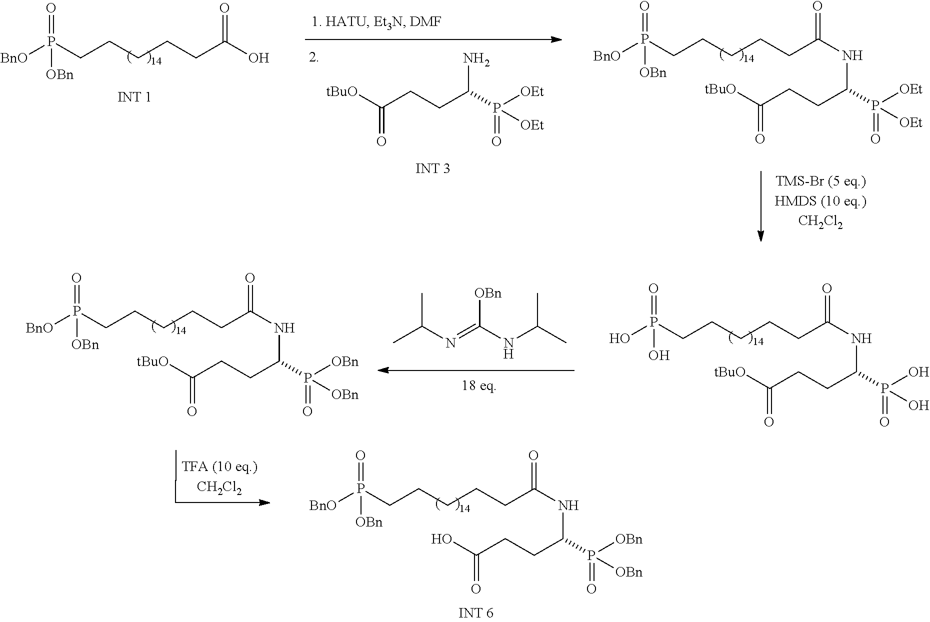

### Example 2

### Synthesis of Common Peptide Backbone

The 39-amino acid peptide backbone is constructed using solid-phase peptide synthesis techniques with diimide, HATU, or HBTU activation for amide linkage synthesis on a Rink resin. Reagent selection varies based on the identity of the amino acids being connected. The R-group of lysine-19 was extended with two PEG₂ amide linkers. The entire backbone is synthesized on the resin before coupling INT 4, INT 5, or INT 6 to the amino terminus of the lysine-bound linker.

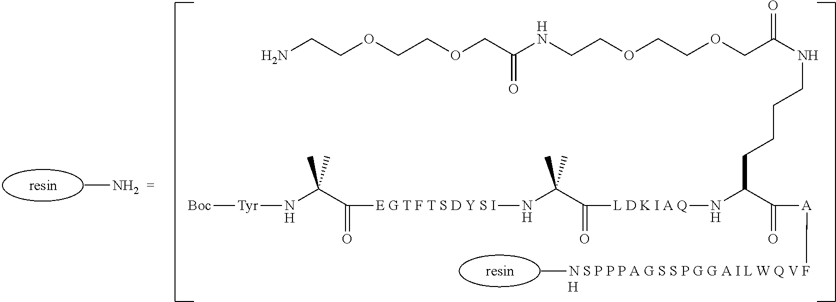

### Example 3

### Synthesis of Compound 4

The peptide backbone is coupled to INT 4 to give resin-bound, protected Compound 4. Cleavage of the resin, protecting groups on the peptide chain, and benzyl esters of INT 4 with TFA provides Compound 4, which is purified through HPLC.

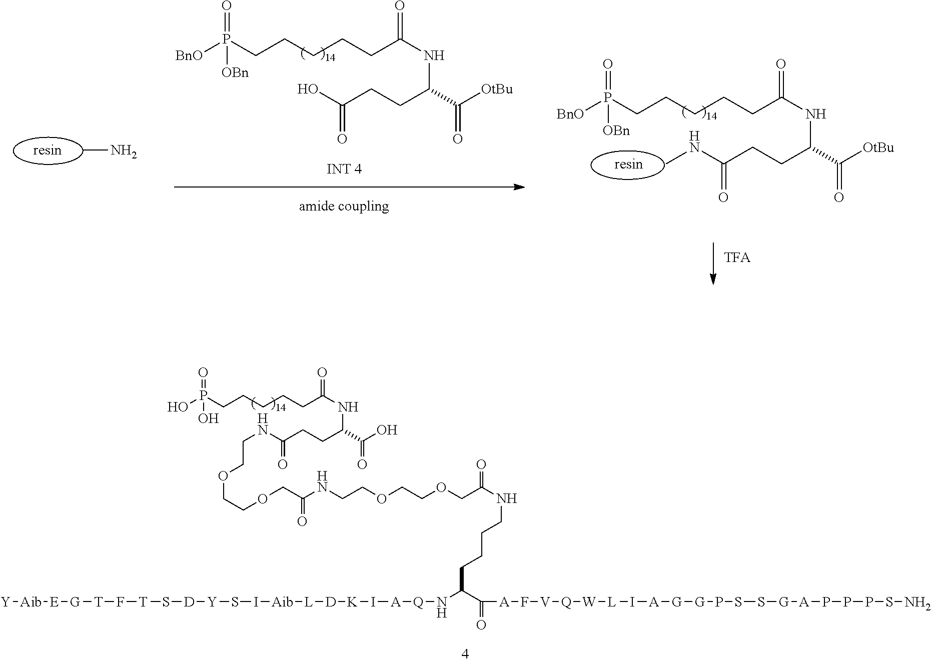

### Example 4

### Synthesis of Compound 8

The peptide backbone is coupled to INT 5 to give resin-bound, protected Compound 8. Cleavage of the resin, protecting groups on the peptide chain, and benzyl esters of INT 4 with TFA provides Compound 8, which is purified through HPLC.

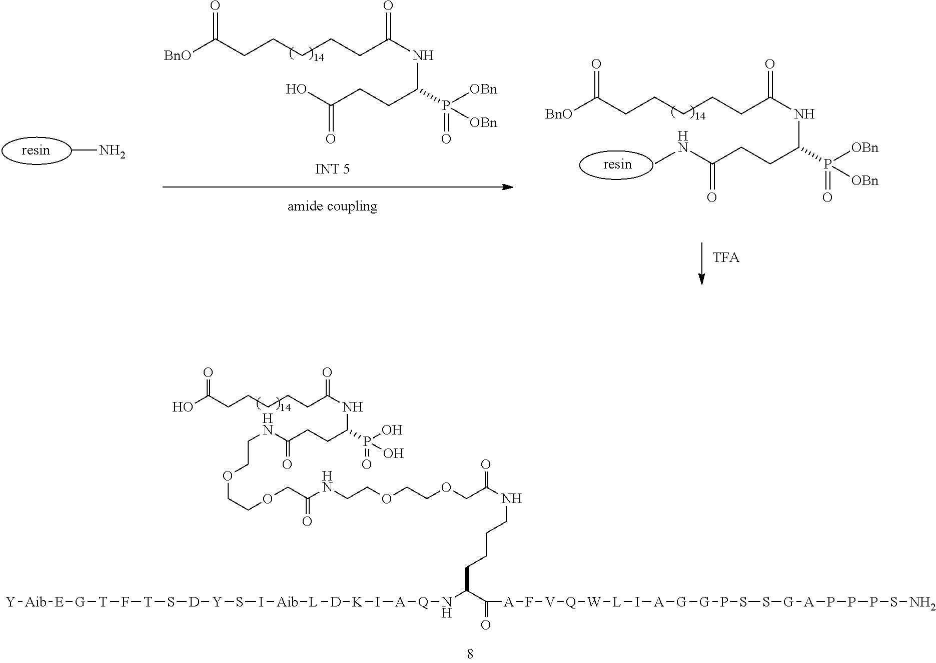

### Example 5

### Synthesis of Compound 12

The peptide backbone is coupled to INT 6 to give resin-bound, protected Compound 12. Cleavage of the resin, protecting groups on the peptide chain, and benzyl esters of INT 4 with TFA provides Compound 12, which is purified through HPLC.

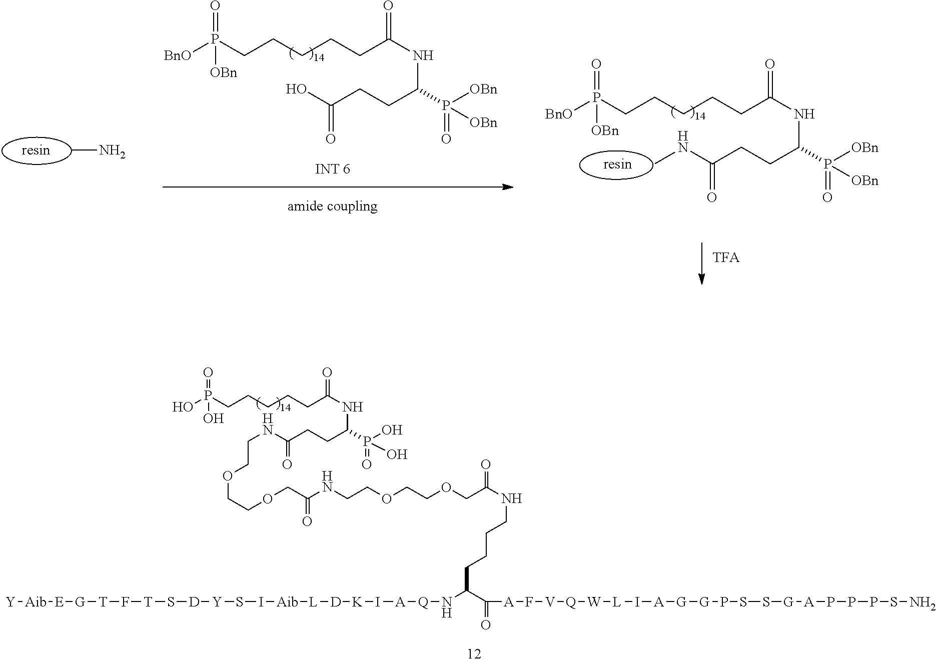

### Example 6

### Pharmacokinetics Studies of Formulations

Subcutaneous (SC) dosing of tirzepatide and Compound 4 in two different formulations was performed with male cynomolgus monkeys. Formulation 1 included the compound in a vehicle of 0.1% bovine serum albumin in phosphate buffered saline solution. Formulation 2 included the compound in 40% propylene glycol and 609% 10 mM pH 6 citrate buffer solution. Assigned dosage groups were administered tirzepatide (0.2 mg/kg) or Compound 4 (0.2 mg/kg) over 21 days. Samples were obtained at 1, 4, 8, 12, 24, 48, 72, 96, 120, 168, 192, 240 and 336 hours after the single dose was administered.

Mean half life values are shown in Table 1. The data shows that administration of Compound 4 in Formulation 1 and Formulation 2 resulted in significantly greater persistence in the blood stream when compared to administration of tirzepatide in the identical formulation. Surprisingly, the half life of Compound 4 was greater in Formulation 2 than Formulation 1. Even more surprisingly, the relative increase in half life of Compound 4 compared to tirzepatide was greater in Formulation 2 than Formulation 1. Formulation 2 resulted in a mean half life of Compound 4 that is twice as long when compared to tirzepatide.

TABLE 1

| | | Half Life |
|---|---|---|
| | Compound ID | (h) |
| Formulation 1 | Tirzepatide | 66.2 |
| | 4 | 106 |
| Formulation 2 | Tirzepatide | 59.8 |
| | 4 | 120 |

Binding assays of tirzepatide and Compound 4 were performed in the presence or absence of 2% human serum albumin (HSA). The ratio of compound-receptor binding with 2% HSA to compound-receptor binding with 0% HSA is listed in Table 2. Tirzepatide has an HSA ratio of 12.8 and 5.82 for the GLP-1 receptor and GIP receptor, respectively. Compound 4 has an HSA ratio of 6.42 and 1.25 for the GLP-1 receptor and GIP receptor, respectively. The larger HSA ratios of tirzepatide when compared to Compound 4 indicates that the binding affinity of tirzepatide for albumin is greater than that of Compound 4.

TABLE 2

| | GLP-1 receptor IC 50 (nM) | | | GIP receptor IC 50 (nM) | | |
|---|---|---|---|---|---|---|
| Sample | 2% | 0% | Ratio of | 2% | 0% | Ratio of |
| ID | HSA | HSA | HSA:No HSA | HSA | HSA | HSA:No HSA |
| TRZ | 3409 | 266 | 12.8 | 1840 | 316 | 5.82 |
| 4 | 988 | 154 | 6.42 | 331 | 264 | 1.25 |

In previous studies, a direct correlation between albumin binding affinity and in vivo half life was observed; compounds having a longer half life displayed greater albumin binding affinity. (Lau, J., et al. J. Med. Chem. 2015, 58, 7370-7380). As described above, the binding affinity of tirzepatide for albumin is greater than that of Compound 4. In contrast, the pharmacokinetic experiments described in this example determined the half life of tirzepatide and Compound 4 to be 66.2 h and 106 h, respectively, in Formulation 1. The half life of tirzepatide and Compound 4 were determined to be 59.8 h and 120 h, respectively, in Formulation 2. The longer half life of Compound 4 in Formulation 1 and Formulation 2 when compared to that of tirzepatide is an unexpected result in view of the previous studies. The smaller HSA ratio of Compound 4 indicates that Compound 4 would be expected to have lower albumin binding affinity and shorter half life relative to tirzepatide. The longer half life for Compound 4 in Formulation 1 and Formulation 2 relative to tirzepatide is opposite of the expected results.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

While the disclosure has been particularly shown and described with reference to a preferred embodiment and various alternate embodiments, it will be understood by persons skilled in the relevant art that various changes in form and details can be made therein without departing from the spirit and scope of the disclosure.

All references, issued patents and patent applications cited within the body of the instant specification are hereby incorporated by reference in their entirety, for all purposes.

Although the disclosure has been described with reference to embodiments and examples, it should be understood that numerous and various modifications can be made without departing from the spirit of the disclosure. Accordingly, the disclosure is limited only by the following claims.

What is claimed is:

1. A pharmaceutical formulation for administration to a subject in need thereof, wherein the pharmaceutical formulation comprises:
at least 10% by weight of propylene glycol; and
a therapeutically effective dosage of a compound having the structure of formula I, or a pharmaceutically acceptable salt thereof, wherein: 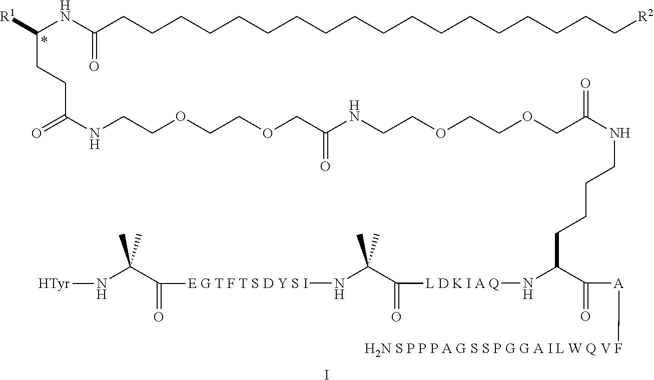
R¹ is selected from the group consisting of —C(═O)(OZ¹), —P(═O)(X)(Y) and a 5-10 membered heteroaryl containing 1-2 heteroatoms selected from N, O and S optionally substituted with 1-2 R⁷ independently selected from halogen, C₁₋₆ alkyl, haloC₁₋₆ alkyl, haloC₁₋₆ alkoxy, —OR⁵, C₃₋₁₀ cycloalkyl, C₆₋₁₀ aryl, 5-10 membered heteroaryl and 5-10 membered heterocyclyl;
R² is selected from the group consisting of —C(═O)OZ²), —P(═O)(X)(Y) and a 5-10 membered heteroaryl containing 1-2 heteroatoms selected from N, O and S optionally substituted with 1-2 R⁷ independently selected from halogen, C₁₋₆ alkyl, haloC₁₋₆ alkyl, haloC₁₋₆ alkoxy, —OR⁵, C₃₋₁₀ cycloalkyl, C₆₋₁₀ aryl, 5-10 membered heteroaryl and 5-10 membered heterocyclyl;
each R⁷ is independently selected from the group consisting of halogen, C₁₋₆ alkyl, haloC₁₋₆ alkyl, haloC₁₋₆ alkoxy, C₁₋₆ alkoxy, C₃₋₁₀ cycloalkyl, C₆₋₁₀ aryl, 5-10 membered heteroaryl and 5-10 membered heterocyclyl;
X and Y each are independently selected from the group consisting of —OR⁴, NR⁵R⁶, C₁₋₆ alkyl and haloC₁₋₆ alkyl;
each R⁴ is independently selected from the group consisting of hydrogen, C₁₋₆ alkyl, haloC₁₋₆ alkyl, C₆₋₁₀ aryloxy and C₆₋₁₀ aryl alkoxy;
each R⁵ is independently hydrogen or C₁₋₆ alkyl;
each R⁶ is independently hydrogen or C₁₋₆ alkyl; and
Z¹ and Z² each are independently selected from the group consisting of hydrogen, C₁₋₆ alkyl, haloC₁₋₆ alkyl, haloC₁₋₆ alkoxy, C₁₋₆ alkoxy, C₃₋₁₀ cycloalkyl and C₆₋₁₀ aryl,
wherein at least one of Z¹ and Z² is not hydrogen.

2. The pharmaceutical formulation of claim 1, wherein the compound has the structure of formula I-a: 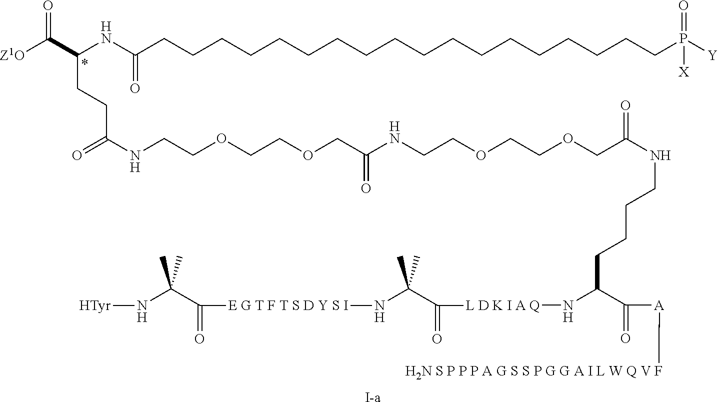
or a pharmaceutically acceptable salt thereof.

3. The pharmaceutical formulation of claim 2, wherein Z¹ is selected from the group consisting of hydrogen, C₁₋₆ alkyl, haloC₁₋₆ alkyl, haloC₁₋₆ alkoxy, C₁₋₆ alkoxy, C₃₋₁₀ cycloalkyl and C₆₋₁₀ aryl; and X and Y each are —OR⁴.

4. The pharmaceutical formulation of claim 2 or 3, wherein Z¹ is selected from the group consisting of hydrogen, haloC₁₋₆ alkoxy and C₁₋₆ alkoxy; and each R⁴ independently is selected from the group consisting of hydrogen, C₆₋₁₀ aryloxy and C₆₋₁₀ aryl alkoxy.

5. The pharmaceutical formulation of any one of claims 2-4, wherein Z¹ is hydrogen and each R⁴ independently is hydrogen or C₆₋₁₀ aryl alkoxy.

6. The pharmaceutical formulation of any one of claims 2-5, wherein each R⁴ is hydrogen.

7. The pharmaceutical formulation of any one of claims 2-6, wherein Z¹ is hydrogen and each R⁴ is hydrogen.

8. The pharmaceutical formulation of claim 1, wherein the compound has the structure of formula I-b: 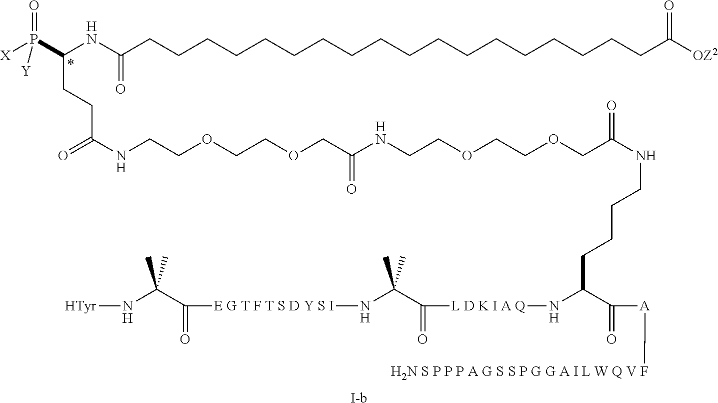
or a pharmaceutically acceptable salt thereof.

9. The pharmaceutical formulation of claim 8, wherein Z² is selected from the group consisting of hydrogen, C₁₋₆ alkyl, haloC₁₋₆ alkyl, haloC₁₋₆ alkoxy, C₁₋₆ alkoxy, C₃₋₁₀ cycloalkyl and C₆₋₁₀ aryl; and X and Y each are —OR⁴.

10. The pharmaceutical formulation of claim 8 or 9, wherein Z² is selected from the group consisting of hydrogen, haloC₁₋₆ alkoxy and C₁₋₆ alkoxy; and each R⁴ independently is selected from the group consisting of hydrogen, C₆₋₁₀ aryloxy and C₆₋₁₀ aryl alkoxy.

11. The pharmaceutical formulation of any one of claims 8-10, wherein Z² is hydrogen and each R⁴ is hydrogen or C₆₋₁₀ aryl alkoxy.

12. The pharmaceutical formulation of any one of claims 8-11, wherein each R⁴ is hydrogen.

13. The pharmaceutical formulation of any one of claims 8-12, wherein Z² is hydrogen and each R⁴ is hydrogen.

14. The pharmaceutical formulation of claim 1, wherein the compound has the structure of formula I-c: 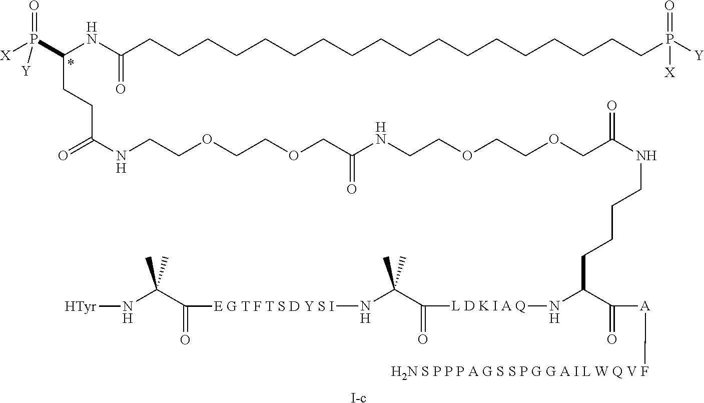
or a pharmaceutically acceptable salt thereof.

15. The pharmaceutical formulation of claim 14, wherein X and Y each are —OR⁴.

16. The pharmaceutical formulation of claim 14 or 15, wherein each R⁴ is independently selected from the group consisting of hydrogen, C₆₋₁₀ aryloxy and C₆₋₁₀ aryl alkoxy.

17. The pharmaceutical formulation of any one of claims 14-16, wherein each R⁴ is hydrogen.

18. The pharmaceutical formulation of claim 1, wherein the compound has the structure selected from the group consisting of: 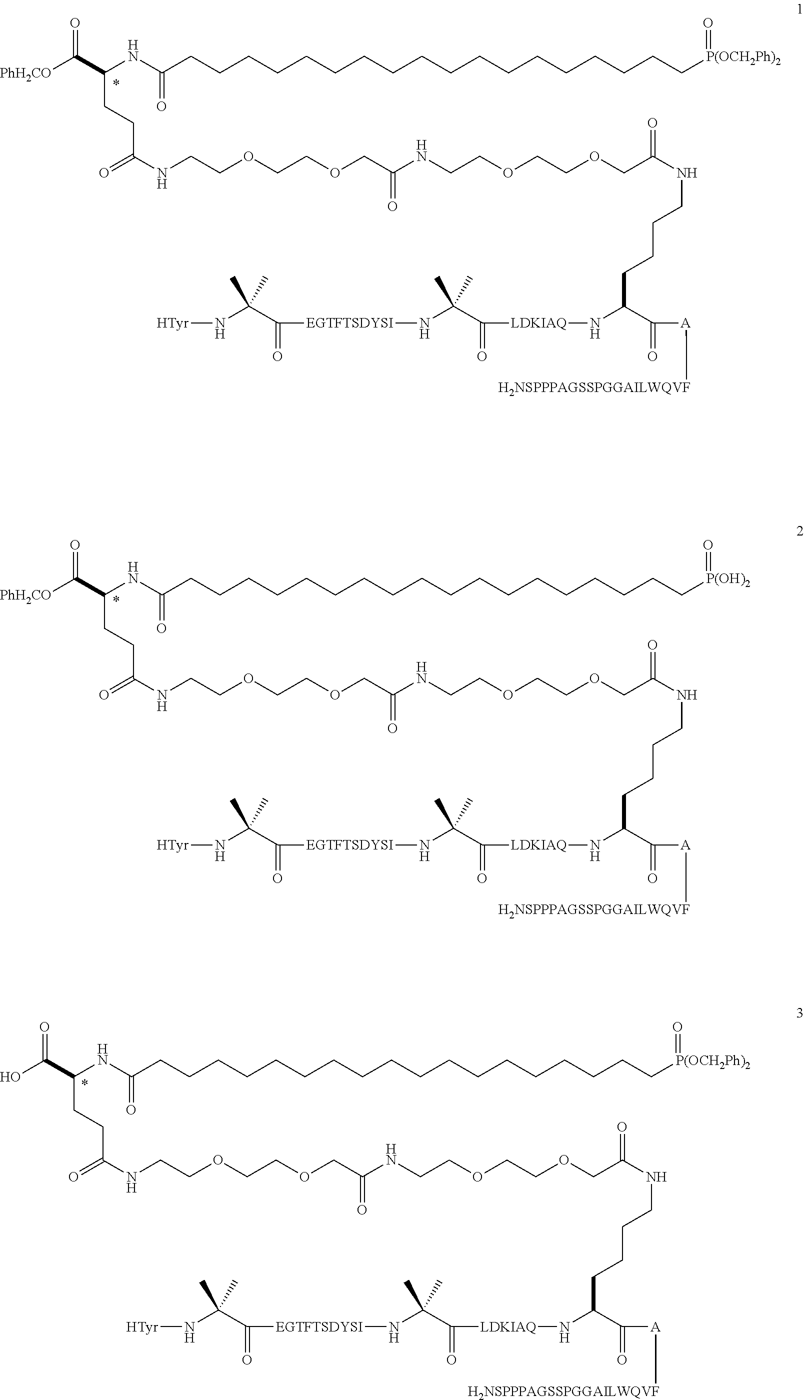 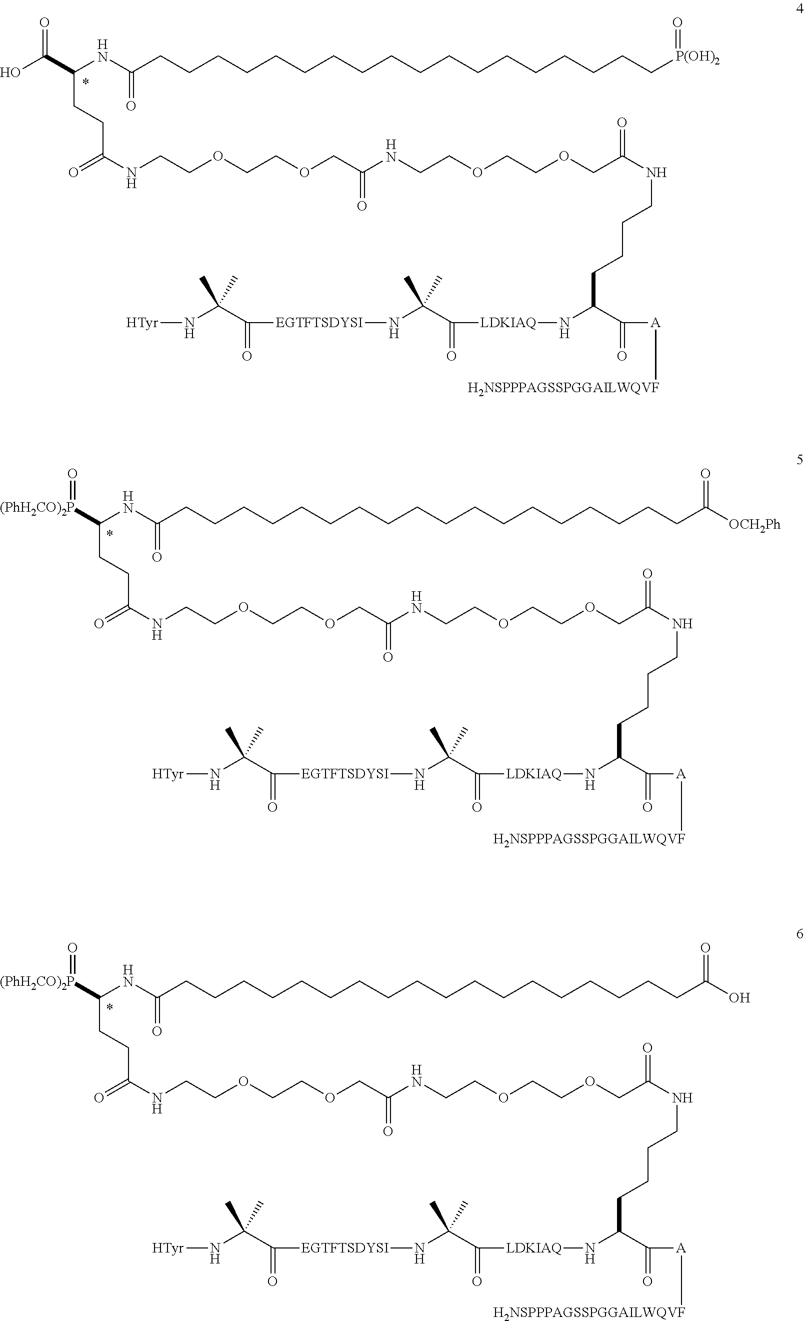 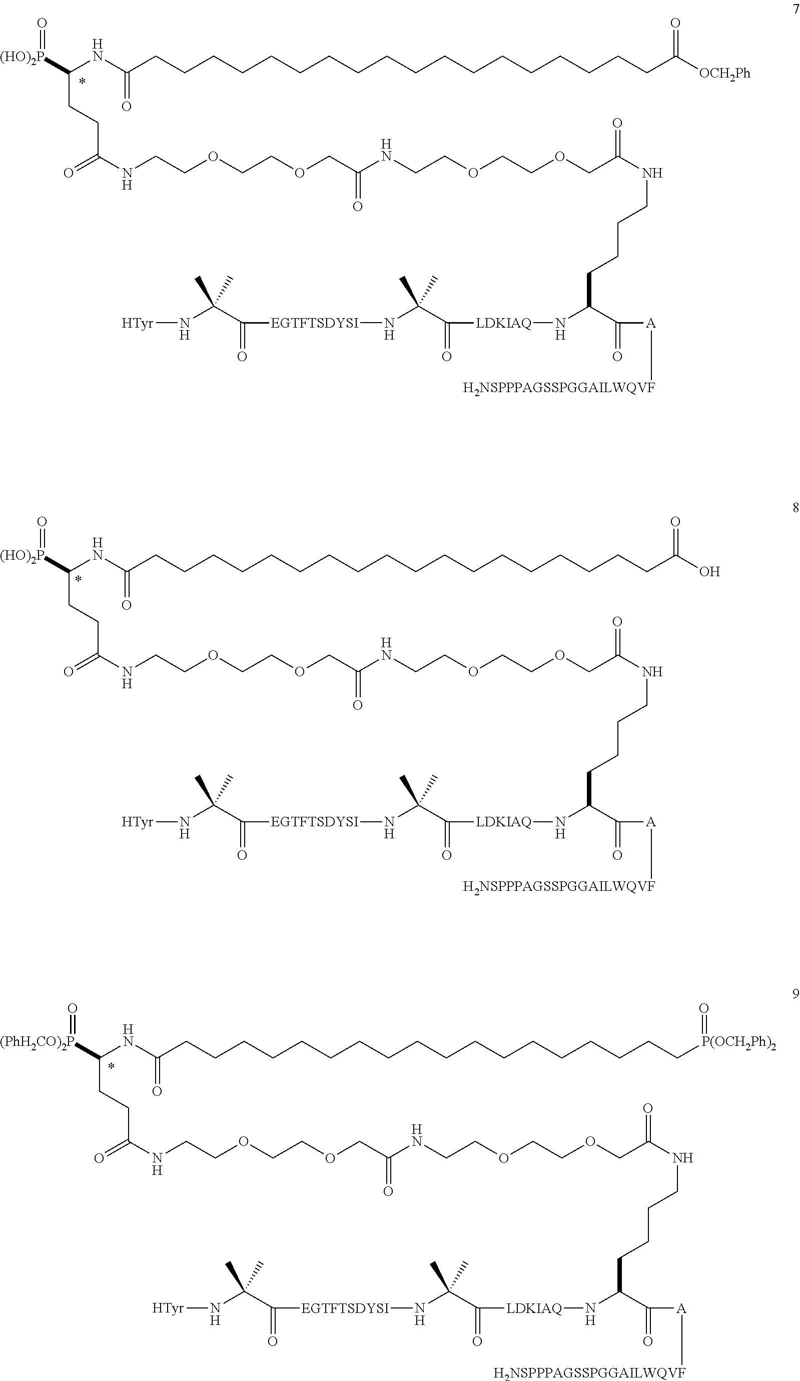 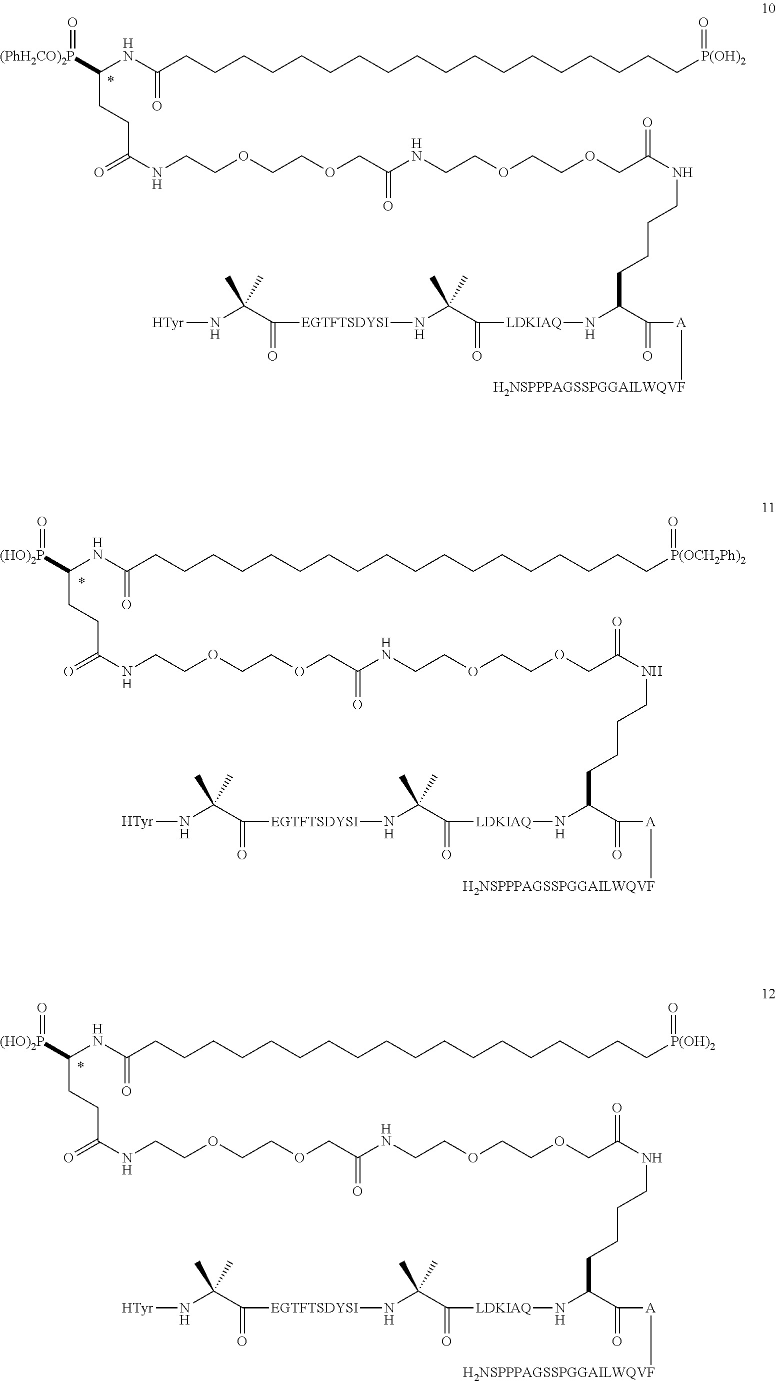
and pharmaceutically acceptable salts thereof.

19. The pharmaceutical formulation of any one of claims 1-18, wherein “*” indicates a chiral carbon with “S” configuration.

20. The pharmaceutical formulation of any one of claims 1-18, wherein “*” indicates a chiral carbon with “R” configuration.

21. The pharmaceutical formulation of any one of claims 1-20, comprising a pharmaceutically acceptable aqueous carrier.

22. The pharmaceutical formulation of any one of claims 1-20, wherein the aqueous carrier is water, an aqueous buffer, or saline.

23. The pharmaceutical formulation of any one of claims 1-22, wherein the propylene glycol is present in the formulation at a weight percentage of equal to or more than about 15%.

24. The pharmaceutical formulation of any one of claims 1-23, wherein the propylene glycol is present in the formulation at a weight percentage from about 15% to 90%.

25. The pharmaceutical formulation of any one of claims 1-24, wherein the propylene glycol is present in the formulation at a weight percentage from about 30% to 50%.

26. The pharmaceutical formulation of any one of claims 1-25, wherein the formulation comprises a buffer.

27. The pharmaceutical formulation of any one of claims 1-26, wherein the buffer comprises tartrate, citrate, acetate, 2-(N-morpholino)ethanesulfonic acid (MES), piperazine-N,N′-bis(2-ethanesulfonic acid (PIPES), 3-(N-morpholino)propanesulfonic acid (MOPS), 2-[[1,3-dihydroxy-2-(hydroxymethyl)propan-2-yl]amino]ethanesulfonic acid (TES), 4-(2-hydroxyethyl)-1-piperazineethanesulfonic acid (HEPES), 3-[N-tris(hydroxymethyl)methylamino]-2-hydroxypropanesulfonic acid (TAPSO), N-[tris(hydroxymethyl)methyl]glycine (Tricine), tris(hydroxymethyl)aminomethane (Tris), 2-(bis(2-hydroxyethyl)amino)acetic acid (Bicine), tris(hydroxymethyl)methylamino]propanesulfonic acid (TAPS), N-cyclohexyl-2-aminoethanesulfonic acid (CHES), phosphate, borate, or any combination of the foregoing.

28. The pharmaceutical formulation of any one of claims 1-27, wherein the buffer is present in the formulation at a weight percentage of equal to or more than about 20%.

29. The pharmaceutical formulation of any one of claims 1-28, wherein the buffer is present in the formulation at a weight percentage from about 20% to 95%.

30. The pharmaceutical formulation of any one of claims 1-29, wherein the buffer is present in the formulation at a weight percentage from about 50% to 70%.

31. The pharmaceutical formulation of claim 26, wherein the buffer comprises citrate.

32. The pharmaceutical formulation of claim 26, wherein the buffer comprises acetate.

33. The pharmaceutical formulation of claim 26, wherein buffer comprises 2-(N-morpholino)ethanesulfonic acid (MES).

34. The pharmaceutical formulation of claim 26, wherein the buffer comprises piperazine-N,N′-bis(2-ethanesulfonic acid (PIPES).

35. The pharmaceutical formulation of any one of claims 1-34, wherein the formulation has a pH from about 2 to 12.

36. The pharmaceutical formulation of any one of claims 1-35, wherein the formulation has a pH from about 3 to 7.

37. The pharmaceutical formulation of any one of claims 1-36, wherein the formulation has a pH from about 4 to 6.8.

38. The pharmaceutical formulation of any one of claims 26-37, wherein the propylene glycol is present in the formulation at a weight percentage of equal to or more than about 15% and the buffer is present in the formulation at a weight percentage of equal to or more than about 20%.

39. The pharmaceutical formulation of claim 38, wherein the propylene glycol is present in the formulation at a weight percentage from about 30% to 50%.

40. The pharmaceutical formulation of claim 38 or 39, wherein the buffer is present in the formulation at a weight percentage from about 20% to 95%.

41. The pharmaceutical formulation of claim 38 or 39, wherein the buffer is present in the formulation at a weight percentage from about 30% to 80%.

42. The pharmaceutical formulation of claim 38 or 39, wherein the buffer is present in the formulation at a weight percentage from about 50% to 70%.

43. The pharmaceutical formulation of any one of claims 1-42, wherein the therapeutically effective dosage is from about 0.01 mg/kg to about 5 mg/kg.

44. The pharmaceutical formulation of any one of claims 1-43, wherein when administered the pharmaceutical formulation has a half life of equal to or greater than about 40 h.

45. The pharmaceutical formulation of any one of claims 1-44, wherein when administered the pharmaceutical formulation has a half life from about 40 h to 300 h.

46. The pharmaceutical formulation of any one of claims 1-45, wherein when administered the pharmaceutical formulation has a half life from about 60 h to 150 h.

47. The pharmaceutical formulation of any one of claims 44-46, wherein the administration is a route of administration selected from the group consisting of enteral, intravenous, oral, intraarticular, intramuscular, subcutaneous, intraperitoneal, epidural, transdermal, and transmucosal.

48. The pharmaceutical formulation of any one of claims 1-47, wherein the pharmaceutical formulation comprises a dosage form selected from a solid form and a liquid form.

49. A method of preventing, treating, or ameliorating one or more fatty liver diseases in a subject, comprising administering a pharmaceutical formulation of any one of claims 1-48, to a subject in need thereof.

50. The method of claim 49, wherein said wherein said fatty liver disease is selected from the group consisting of steatosis, non-alcoholic steatohepatitis and non-alcoholic fatty liver disease.

51. The method of claim 49 or 50, wherein said administration of said pharmaceutical formulation results in the prevention, treatment, or amelioration, of a fibrosis, fibrotic condition, or fibrotic symptoms.

52. The method of any one of claims 49-51, wherein said administration of said pharmaceutical formulation results in the reduction in the amount of extracellular matrix proteins present in one or more tissues of said subject.

53. The method of any of claims 49-52, wherein said administration of said pharmaceutical formulation results in the reduction in the amount of collagen present in one or more tissues of said subject.

54. The method of claim 53, wherein said administration of said pharmaceutical formulation results in the reduction in the amount of Type I, Type Ia, or Type III collagen present in one or more tissues of said subject.

55. A method of preventing, treating, or ameliorating one or disease or disorders in a subject, comprising administering a pharmaceutical formulation of any one of claims 1-48 to a subject in need thereof, wherein said disease or disorder is liver fibrosis, renal fibrosis, biliary fibrosis, pancreatic fibrosis, nonalcoholic steatohepatitis, non-alcoholic fatty liver disease, chronic kidney disease, diabetic kidney disease, primary sclerosing cholangitis, primary biliary cirrhosis, or idiopathic fibrosis.

56. The method of claim 55, wherein said disease or disorder is nonalcoholic steatohepatitis, non-alcoholic fatty liver disease, chronic kidney disease, diabetic kidney disease, primary sclerosing cholangitis, or primary biliary cirrhosis.

57. The method of claim 55, wherein the route of administration is selected from the group consisting of: enteral, intravenous, oral, intraarticular, intramuscular, subcutaneous, intraperitoneal, epidural, transdermal, and transmucosal.
